(19) United States
(12) Patent Application Publication
(10) Pub. No.: US 2011/0015484 A1
(43) Pub. Date: Jan. 20, 2011

(54) ENDOSCOPIC ROBOTIC CATHETER SYSTEM

(76) Inventors: Jeffrey B. Alvarez, San Mateo, CA (US); Federico Barbagli, San Francisco, CA (US); Christopher R. Carlson, Menlo Park, CA (US); Gregory J. Stahler, San Jose, CA (US); Neal A. Tanner, Mountain View, CA (US)

Correspondence Address: VISTA IP LAW GROUP LLP, 12930 Saratoga Avenue, Suite D-2, Saratoga, CA, 95070, US

(21) Appl. No.: 12/504,569

(22) Filed: Jul. 16, 2009

(51) Int. Cl. A61B 1/04; A61B 1/06; A61M 25/00

(52) U.S. Cl. 600/109; 600/182; 604/528

(57) ABSTRACT

A robotic catheter system includes a controller with a master input device. An instrument driver is in communication with the controller and has a elongate instrument interface including a plurality of instrument drive elements responsive to control signals generated, at least in part, by the master input device. An elongate instrument has a base, distal end, and a working lumen, wherein the guide instrument base is operatively coupled to the guide instrument interface. The elongate instrument preferably comprises and/or defines other lumens to accommodate instruments such as an optics bundle, a light bundle, a laser fiber, and flush irrigation. The working lumen preferably is configured to accommodate a grasping or capturing tool, such as a collapsible basket or grasper, for use in procedures such as kidney stone interventions. The elongate instrument includes a plurality of instrument control elements operatively coupled to respective drive elements and secured to the distal end of the instrument. The instrument control elements are axially moveable relative to the guide instrument such that movement of the guide instrument distal end may be controlled by the master input device.

### FIELD OF THE INVENTION

The invention relates generally to robotically controlled systems, such as telerobotic surgical systems, and more particularly to a robotic catheter system for performing minimally invasive diagnostic and therapeutic procedures.

### BACKGROUND

Robotic surgical systems and devices are well suited for use in performing minimally invasive medical procedures, as opposed to conventional techniques wherein the patient's body cavity is open to permit the surgeon's hands access to internal organs. For example, there is a need for a highly controllable yet minimally sized system to facilitate imaging, diagnosis, and treatment of tissues which may lie deep within a patient, and which may be preferably accessed only via naturally-occurring pathways such as blood vessels, gastrointestinal tract, or urinary tract.

### SUMMARY

One embodiment is directed to a robotic catheter system, comprising a robotically steerable catheter instrument comprising a housing and an elongate flexible body, the elongate flexible body defining one or more instrument lumens longitudinally therethrough; an electromechanical instrument driver comprising a housing, a plurality of motors, and an instrument interface configured to removably engage the catheter instrument housing and transfer a plurality of steering actuations to the catheter instrument when engaged thereto; an elongate medical instrument slidably positioned through one of the one or more instrument lumens; an electromechanical device actuation assembly coupled to the proximal end of the elongate medical instrument and configured to actuate the medical instrument independently of steering actuations transferred to the robotically steerable catheter instrument; and a controller configured to operate the instrument driver and device actuation assembly to respectively steer the catheter instrument and actuate the elongate medical instrument subject to commands imparted by an operator to a master input device operably coupled to the controller. The one or more instrument lumens may be configured to separately and simultaneously accommodate an optics bundle, a light bundle, and a laser fiber, and to define a flush channel. The instrument housing may comprise an optics bundle connection interface. The instrument housing may comprise a light bundle connection interface. The instrument housing may comprise a laser fiber coupling interface. The instrument housing may comprise a flush channel supply interface. In one embodiment, one of the one or more instrument lumens may be sized to accommodate slidable coupling of a collapsible basket tool. An optics bundle connection interface may comprise a translucent light transmission interface between fibers comprising the optics bundle in the housing and fibers comprising an external optics bundle coupled to a video processing device. An optics bundle connection interface may comprise a translucent light transmission interface between fibers comprising the optics bundle in the housing and a digital image capture chip. The elongate flexible body may comprise a substantially square cross sectional shape having corners. The corners may be rounded in shape. The elongate flexible body may comprise a polymeric coextrusion. The elongate flexible body may define four integrated instrument lumens arranged immediately adjacent the corners. The elongate flexible body may define four control wire lumens arranged between the four integrated instrument lumens. The working instrument lumen may have a diameter which is greater than that of the integrated instrument lumens, and the control wire lumens may have a diameter which is smaller than that of the integrated instrument lumens. The elongate body may have a first portion with a first flexibility and a second portion extending distally from the first portion, the second portion having a second flexibility substantially greater than the first flexibility. The catheter system may further comprise a flexible sheath body defining a working lumen sized to accommodate slidable engagement of the elongate flexible body, and an outer cross sectional shape that is substantially circular. The shape of the working lumen of the sheath body and outer shape of the elongate flexible body may be jointly configured to limit rotational movement of the elongate flexible body relative to the sheath body. In one embodiment, the device actuation assembly may be configured to controllably insert and retract the medical instrument relative to the catheter instrument. The device actuation assembly may comprise two or more pinch roller wheels movably coupled to the medical instrument. The device actuation assembly may comprise a lead screw assembly coupled to the medical instrument. The device actuation assembly may comprise a recirculatory tension member assembly movably coupled to the medical instrument. The medical instrument may be selected from the group consisting of a laser fiber, a guidewire, a collapsible basket tool, a flexible needle, a grasper, and a balloon tool. In one embodiment, the device actuation assembly may be configured to controllably roll the medical instrument relative to the catheter instrument. The device actuation assembly may comprise two surfaces configured to engage opposite sides of the medical instrument, and to roll the medical instrument as at least one of such surfaces is moved relative to each other through a motion plane substantially parallel to an axis of roll motion defined as the medical instrument is rolled. In one embodiment, one of the two surfaces may be substantially planar. In another embodiment, one of the two surfaces may comprise a pinch roller wheel surface. The device actuation assembly may comprise a rolling subassembly that is coupled to a coupling element configured to removably and fixedly couple to the medical instrument, and wherein the rolling subassembly is rotatably coupled to a base element configured to not rotate relative to the instrument driver. The medical instrument may be selected from the group consisting of a laser fiber, a guidewire, a collapsible basket tool, a flexible needle, a grasper, and a balloon tool. In one embodiment, the device actuation assembly may comprise an elongate tension element operably coupled between an actuator coupled to a housing of the device actuation assembly and a distally operable element of the medical device such that activation of the actuator creates tension in the tension element and a pulling actuation of the distally operable element. The distally operable element may be selected from the group consisting of a grasper closure element, a grasper opener element, a collapsible basket collapse element, and a collapsible basket expand element. In another embodiment, the device actuation assembly may comprise an elongate compression element operably coupled between an actuator coupled to a housing of the device actuation assembly and a distally operable element of the medical device such that activation of the actuator creates compression in the tension element and a pushing actuation of the distally operable element. The distally operable element may be selected from the group consisting of a grasper closure element, a grasper opener element, a collapsible basket collapse element, and a collapsible basket expand element. The device actuation assembly may comprise an injection pump fluidly coupled with one of the lumens defined through the elongate flexible body and configured to inject or suction out fluid through said lumen. The injection pump may comprise a piston operably coupled to an actuator and movably coupled to a fluid reservoir housing. The injection pump may comprise a reservoir configured to contain an injectible fluid selected from the group consisting of liquid contrast agent, saline, and liquid therapeutics. The medical instrument may comprise a balloon tool or a flexible needle. In one embodiment, the medical instrument may have a proximal end comprising a manual control interface, wherein the device actuation assembly comprises one or more fittings configured to removably couple to one or more portions of the manual control interface to substantially reproduce relative motions of such portions when operated manually. At least one of said one or more fittings may comprise a pin array. At least one of said one or more fittings may comprise a post array. At least one of said one or more fittings may be coupled to a motion subassembly selected from the group consisting of an assembly of one or more leadscrews and a recirculatory tension member assembly. At least one of said one or more fittings may be coupled to an assembly of two lead screws configured to move orthogonally relative to each other. At least one of the one or more fittings may be configured to be electromechanically rotated relative to other portions of the manual control interface. The device actuation assembly may comprise a torquable coupling configured to allow for misalignment between an axis of rotation of a rotation actuator providing electromechanical rotation and an axis of rotation of the fitting configured to be electromechanically rotated. The torquable coupling may comprise a Schmidt type coupling.

### BRIEF DESCRIPTION OF THE DRAWINGS

FIG. 1 illustrates a system comprising an operator workstation, an instrument driver, an elongate steerable instrument, and other components in accordance with one embodiment of the subject invention.

FIG. 2 illustrates an alternative configuration wherein an articulated mounting system couples an instrument driver to an operating table.

FIGS. 3A-3D illustrate additional embodiments of instrument driver support structures, each of such embodiments having a mobile base.

FIGS. 4A-4L depict aspects of embodiments of an elongate steerable catheter configuration suitable for use with the subject system.

FIG. 5 depicts a lockable elongate member infrastructure suitable for use in various embodiments of the present invention.

FIGS. 6A-6F illustrate various embodiments of a diagnostic and/or interventional navigation of components in accordance with the present invention toward the kidney through the urethra, bladder, and ureters.

FIGS. 6G-6N illustrate various embodiments of an interventional navigation of the subject system into one of the calices of the kidney to destroy a kidney stone, in accordance with the present invention.

FIGS. 6O-6U illustrate various embodiments of an interventional navigation of the subject system into one of the inferior calices of the kidney to destroy a kidney stone, in accordance with the present invention.

FIG. 6S illustrates one embodiment of a method for conducting a kidney stone intervention in accordance with the present invention.

FIGS. 7A-7D illustrate embodiments for actuating or inserting/retracting various types of instruments operatively coupled with a steerable catheter system.

FIGS. 8A and 8B illustrate an embodiment for imparting roll or rotational actuation to various types of instruments operatively coupled with a steerable catheter system.

FIGS. 9A and 9B illustrate another embodiment for imparting roll or rotational actuation to various types of instruments operatively coupled with a steerable catheter system.

FIGS. 10A and 10B illustrate embodiments for imparting distal end effector actuation for an elongate instrument operatively coupled with a steerable catheter system.

FIGS. 11A and 11B illustrate embodiments wherein fluid actuation may be utilized in injection and expandable balloon related therapies or diagnostics.

FIGS. 12A-12C illustrate three embodiments for electromechanically actuating a proximal manipulation interface configured for manual actuation.

### DETAILED DESCRIPTION

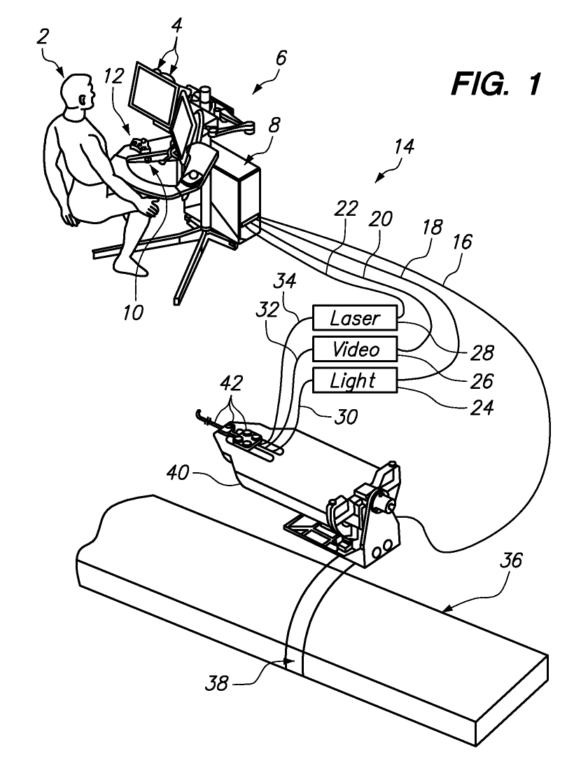

Referring to FIG. 1, a system (14) is depicted wherein an operator (2) is seated at an operator workstation (6) in a position such that he has access to one or more displays (4), in addition to one or more input devices, such as a master input device (10) and an operator button console or pendant (12). A computing system or controller (8) comprising a processor is operably coupled via a cable (16) to a robotic instrument driver (40), which is coupled to an operating table (36) with a fixed mounting member (38). Similar systems have been described, for example, in U.S. patent application Ser. Nos. 11/073,363; 11/179,007; 11/176,598; 11/176,957; 11/481,433; 11/331,576; 11/637,951; 11/640,099; 11/678,001; 11/690,116; 11/804,585; 11/829,076; 11/833,969; 11/852,255; 11/906,746; 11/972,581; 12/032,626; 12/398,763; each of which is incorporated by reference in its entirety into this patent application.

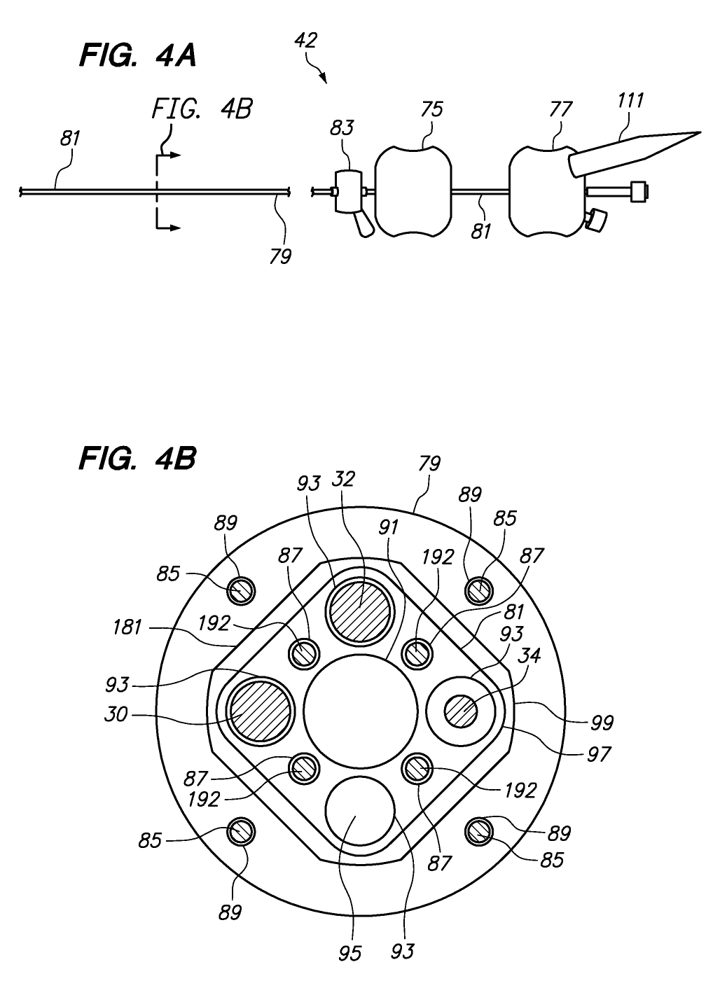

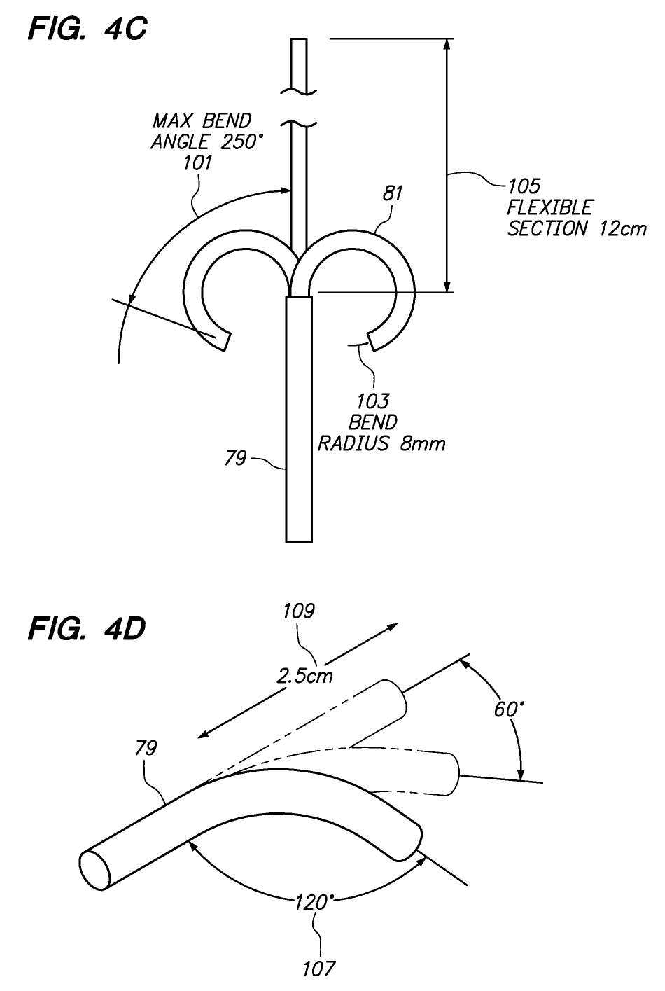

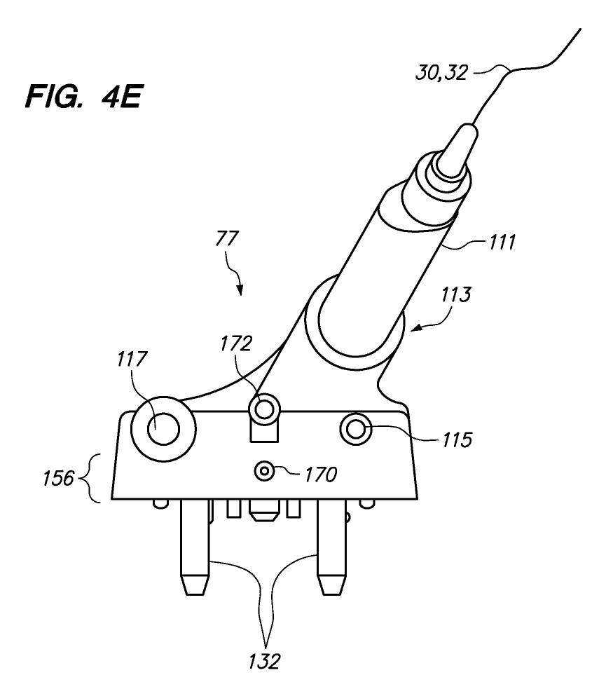

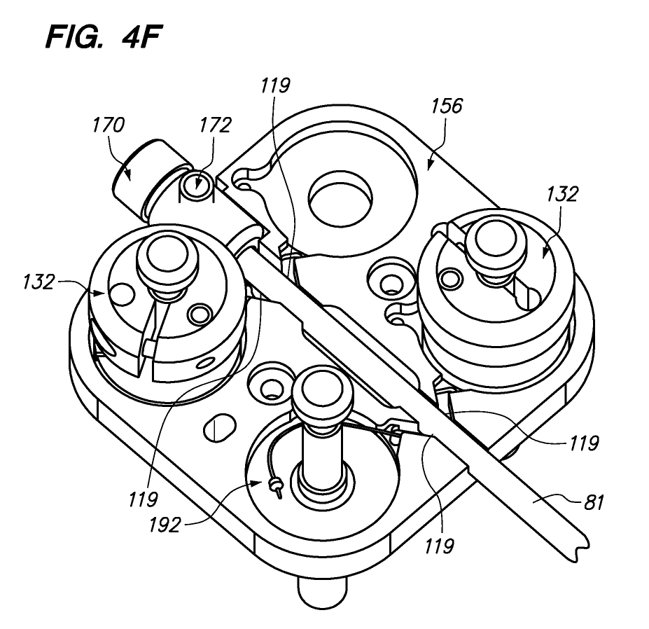

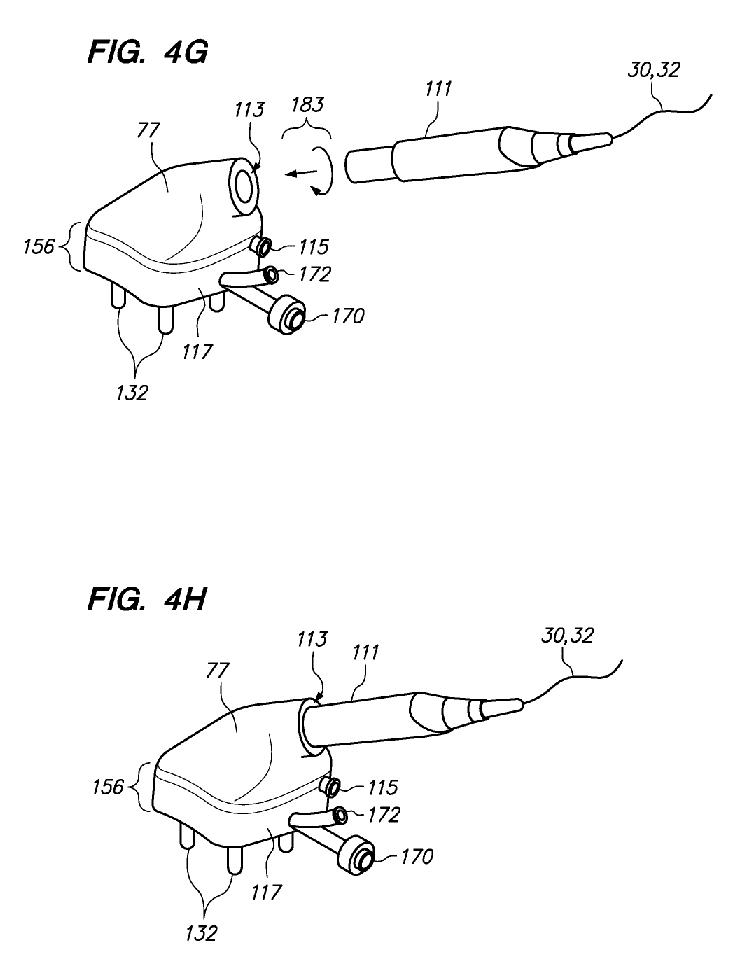

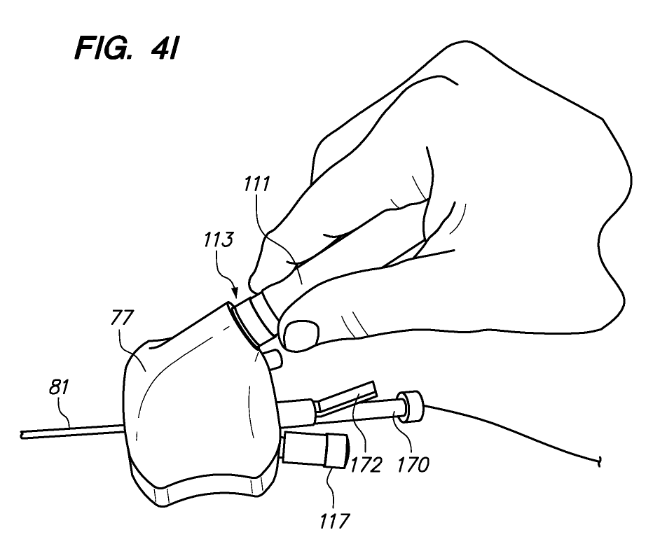

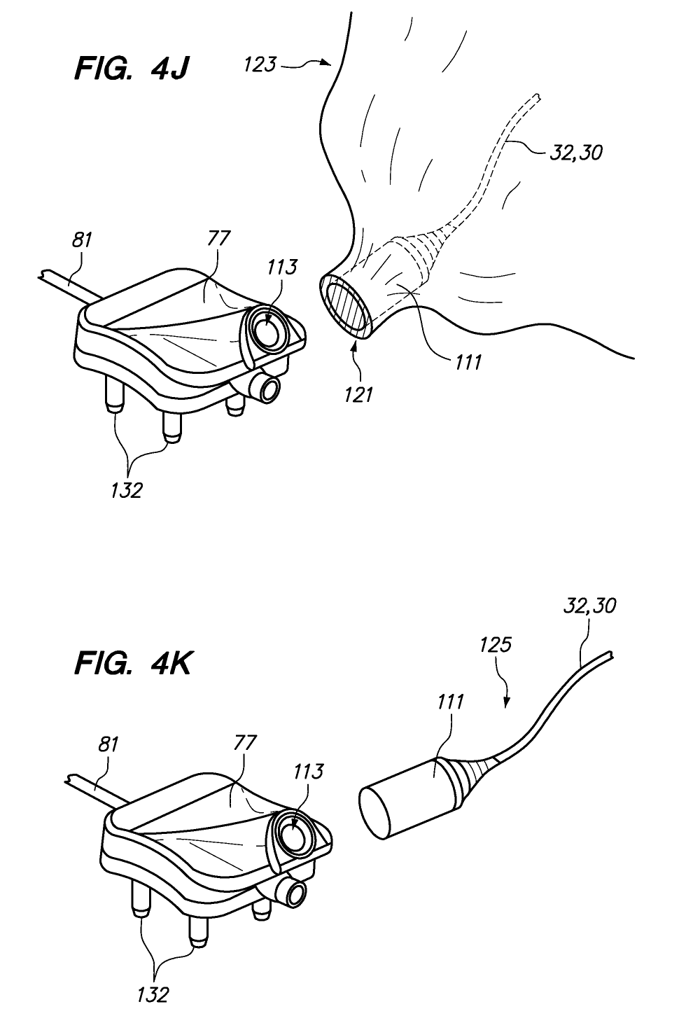

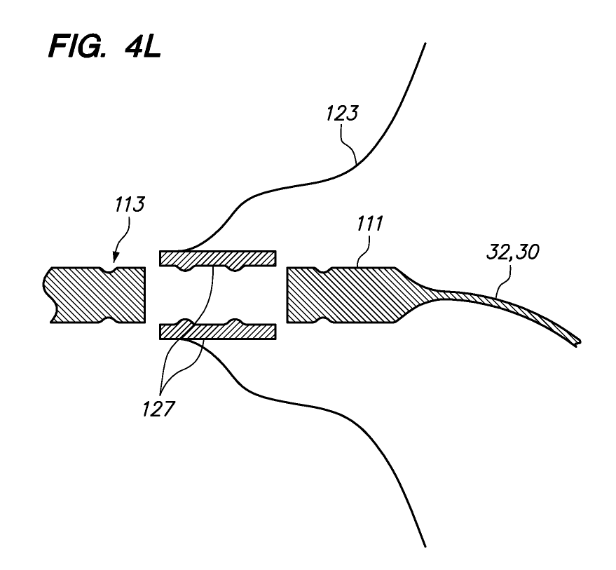

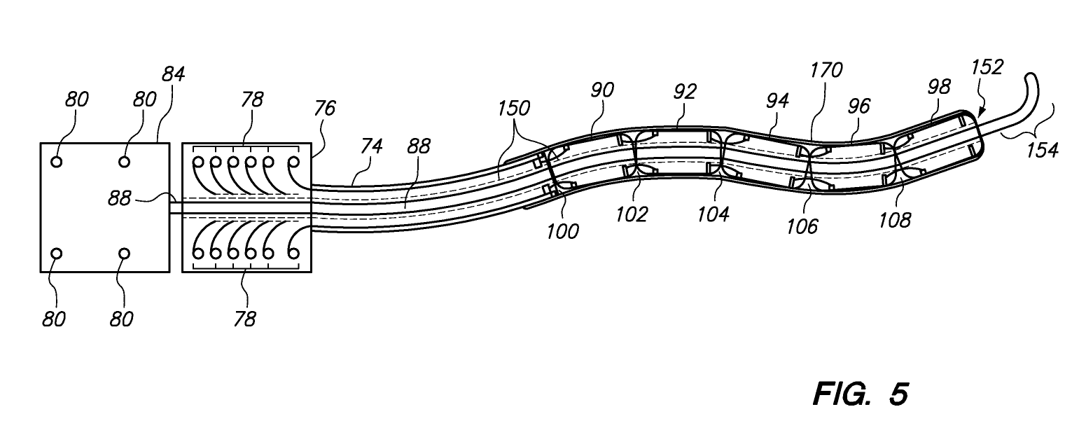

Referring again to FIG. 1, the computing system is operably coupled to a laser therapy system (28), a video system (26), and a lighting system (24) configured to provide endoscopic lighting for the video system (26) by respective cables (22, 20, 18) connecting such systems to the computing system (8). The computing system, via such couplings, is configured to control lighting, video image capture, and laser energy emission, preferably in response to commands input by the operator (2) to interfaces such as the pendant (12) or master input device (10) at the operator workstation (6). Other input devices, such as a foot pedal (not shown), may also be operably coupled to the computing system (8) to enable an operator (2) to execute commands, such as video capture, laser energy emission, and/or lighting, via such input. The laser system (28) is operably coupled to the depicted robotic instrument assembly (42) via a laser energy transmission fiber assembly, or “laser fiber”, (34) while the video system (26) is operably coupled to the instrument assembly (42) via an optics bundle (32) comprising a plurality of optical transmission fibers. The lighting system (24) is similarly operably coupled to the robotic instrument assembly (42) via a light transmission bundle (30) preferably comprising optical transmission fibers. Further details regarding such operable couplings are described in reference to FIGS. 4A-4L.

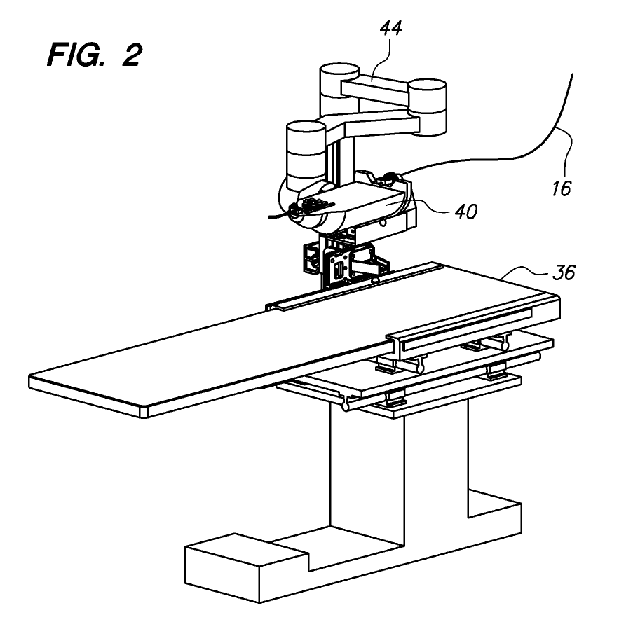

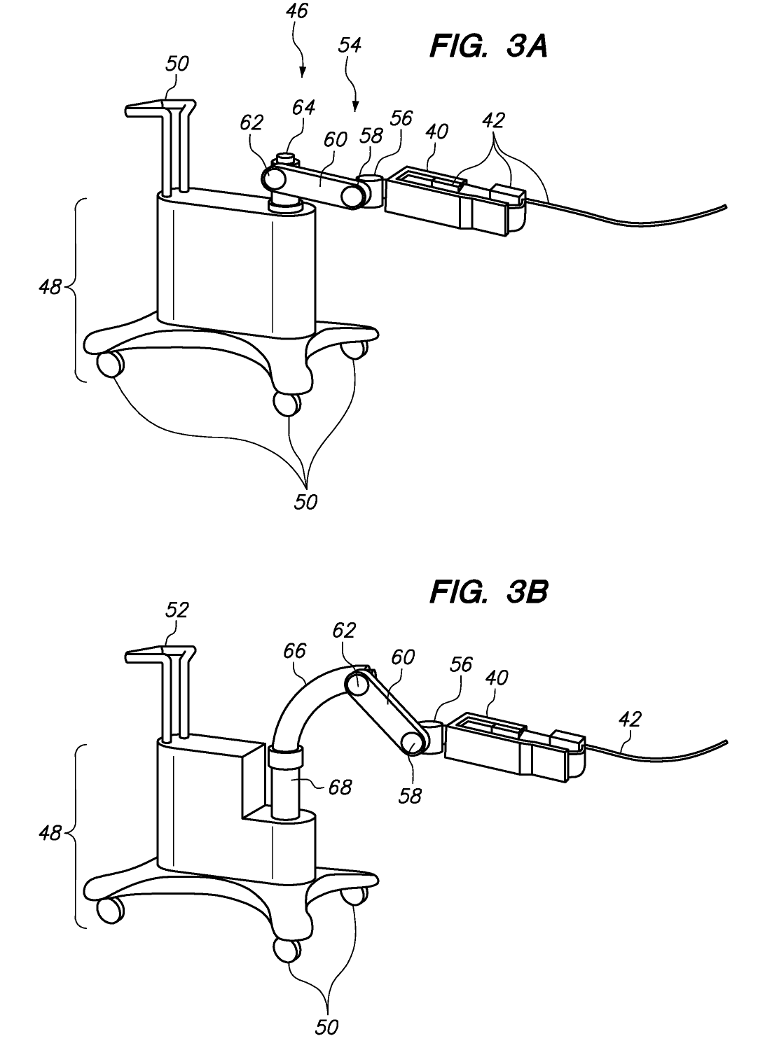

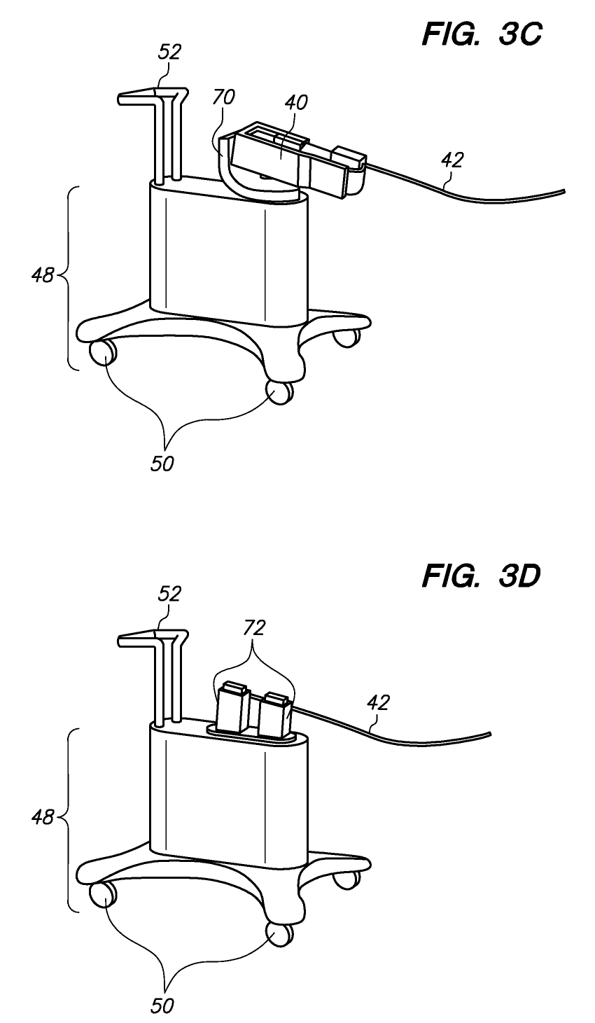

Referring to FIG. 2, another embodiment of an instrument driver (40) mounting structure (44) is depicted coupling the instrument driver (40) to an operating table (36) in an adjustable configuration such that a plurality of revolute joints assist in providing adjustable degrees of freedom. While such a configuration may be desirable for diagnostic and/or interventional procedures wherein a frontal transcutaneous access is to be used, procedures wherein the entry point is at a different orientation, such as through the urethra of a patient lying flat on their back on an operating table (36), may be more desirably accessed using setup structures such as those depicted in FIGS. 3A-3D.

Referring to FIG. 3A, an instrument driver (40) having a robotic catheter instrument assembly (42) coupled thereto is depicted coupled to a mobile setup structure base (48) comprising lockable wheels (50) configured to facilitate movement of the apparatus (46) around the operating room using the depicted handle (52), as well as stability when the wheels (50) are locked into position. The instrument driver (40) is coupled to the mobile base (48) by a mounting frame comprising a distal member (56) coupled to the instrument driver either fixedly or rotatably with a lockable joint (not shown). The distal member (56) is rotatably and lockably coupled to a middle member (60) with a distal member revolute joint (58). The proximal end of the middle member is rotatably and lockably coupled to a proximal member with a proximal member revolute joint (62). The proximal member (64) may be configured to lockably rotate or roll relative to the mobile base (48). Any of these lockable joints may be manually or electromechanically locked, such as by one or more electronic brakes which may be controlled utilizing the controller within the computing system (8) of the operator workstation (6), subject to operator commands at the input devices, or automatically triggered braking conditions, such as by safety logic operated by the computing system (8). Referring to FIG. 3B, another embodiment of a mobile instrument driver (40) mounting system is depicted, this embodiment comprising an arcuate member (66) rotatably coupled to the proximal end of the middle member with a lockable revolute joint (62). The version of the mobile base (48) depicted in FIG. 3B shows an electromechanically lockable roll joint (68) comprising an electric brake. The arcuate member (66) may be desired in certain scenarios wherein clearance and slight elevation provided by the geometry of such arcuate member (66) is important. In other embodiments, the lockable joints described in reference to FIGS. 3A and 3B may not be lockable, but merely joints which are maintained in position via motors (for example, including gravity compensation systems), timing belts, springs, and the like—or in another embodiment, not maintained strictly in position, wherein continual freedom of motion is desired in a certain scenario.

Referring to FIG. 3C, another embodiment of a mobile instrument driver (40) mounting system is depicted, this embodiment comprising a simple coupling structure (70) coupling the instrument driver (40) to the mobile base (48). The simple coupling structure may be a simple fixture without degrees of freedom, or may allow for rotation of the simple coupling structure relative to the mobile base (48) and/or instrument driver (40).

Referring to FIG. 3D, an embodiment is depicted wherein electromechanical instrument driver components (72), such as motors and instrument interfaces, have been mechanically integrated into a mobile base (48). With such embodiment, the instrument (42) is mounted directly to the mobile base assembly.

Referring to FIGS. 4A-4L, aspects of an elongate steerable instrument assembly (42) are described, such assembly being configured for endoscopic diagnosis and/or intervention in an environment wherein direct optical visualization (for example, with an optical image capture device such as a fiberscope or camera chip) is desired, such as with kidney stone interventions using trans-urethral endolumenal access.

Referring to FIG. 4A, an instrument assembly (42) is depicted comprising an inner elongate member, or “guide member”, (81) proximally coupled to a specialized inner instrument base housing (77) which is removably coupleable to an image capture device member (111) preferably comprising a camera chip (not shown). The midsection and distal portion of the inner elongate member (81) are shown slidably coupled and inserted through a working lumen defined through an outer elongate member, or “sheath member”, (79). Also depicted are the outer instrument base housing (75) and a clamp (83) configured to assist with coupling to aspects instrument driver (40—such as that shown in FIG. 1). FIG. 4B is a cross sectional view of the instrument assembly (42) depicted in FIG. 4A. Referring to FIG. 4B, the inner elongate member (81) is threaded through a working lumen (181) defined by the outer elongate member (79). The geometric interaction of the outer elongate member working lumen (181), having a substantially square cross sectional shape with rounded corner surfaces (99), and the outer shape of the inner elongate member (81), which in the depicted embodiment has a square cross sectional outer shape with rounded corners (97), is designed to allow for slidable coupling of the two elongate members (for example, to allow insertion of one relative to the other without a great degree of load applied), while also preventing relative rolling, or rotation, of the two elongate members relative to each other—at least in the areas where they are coupled.

Referring again to FIG. 4B, a relatively complex embodiment is shown for illustrative purposes, wherein the outer elongate instrument member (79) defines four lumens (89) for four control elements (85), such as metallic, semi-metallic, polymeric, or natural pull or pushwires, to enable relatively sophisticated steering of the outer elongate instrument member (79), when such control elements (85) are coupled to a distal portion of the outer elongate instrument member (79), and also coupled to actuator motors within an instrument driver (40) via a mechanical interfacing with rotatable members coupled to the outer instrument base housing (75), as described in the aforementioned incorporated by reference applications. In other words, in one embodiment, the outer instrument may comprise a 4-wire electromechanically steerable sheath instrument capable of omnidirectional steering (for example, when three or four wires terminate at the same position distally), and capable of more complex shapes when one or more wires terminate more proximally than others. Preferably each wire is actuated utilizing an independently operable motor assembly in the instrument driver (40). In other embodiments, such as the embodiments described in the aforementioned incorporated by reference applications, the outer instrument may be much more simple—for example, with only one, two, or even zero control elements. The outer (79) and inner (81) elongate instrument members may comprise polymeric coextrusions.

Referring again to FIG. 4B, the depicted embodiment of the inner elongate instrument member is also relatively sophisticated, defining four instrumentation lumens (93) and a central, larger diameter, working lumen (91) preferably substantially aligned with the longitudinal axis of the inner elongate member (81) and sized to accommodate desired working tools, such as a mini-grasper tool, such as those available from suppliers such as Novare, Inc., or a collapsible basket tool, such as those available from suppliers such as Boston Scientific, Inc. Like the depicted embodiment of the outer elongate instrument member (79), the inner elongate instrument member (81) comprises four control elements (192), such as pushwires and/or pullwires made from metallic, semimetallic, polymeric, or natural materials, threaded through four control element lumens (87). As described above in reference to the outer elongate member (79), this embodiment may be omnidirectionally steerable and/or capable of complex curvatures, via operable coupling of such control elements (191) between distal portions of the inner elongate member (81) and actuation motors within an instrument driver (40). In other embodiments, a more simple configuration comprising one, two, or three control elements (192) may be desired.

Referring again to FIG. 4B, the four instrumentation lumens (93) defined within the depicted embodiment of the inner elongate instrument member (81) are configured to accommodate relatively fixed (in other words, the lumens are large enough to accommodate assembly of the instrument, but small enough to provide a relatively close fit thereafter to prevent significant relative motion) positioning of a light bundle (30) and video/optics bundle (32). Another instrumentation lumen (93) is more loosely and slidably coupled to a laser fiber (34), to allow for relative insertion, retraction, and sometimes roll (depending upon the curvature of the overall assembly) intraoperatively. The fourth instrumentation lumen (93) may be utilized as a saline or other fluid (for example, a contrast agent or medicinal fluid) infusion or flush channel (95) for intraoperative use. Referring to FIG. 4C, in one embodiment, it is desirable that about twelve centimeters of a more flexible, steerable distal portion (105) of the inner elongate instrument member (81) be able to protrude out the distal end of the outer elongate instrument member (79), and that the inner elongate instrument member (81) be capable with such protrusion of forming a bend radius (103) of approximately eight millimeters, with a maximum bend angle (101) of approximately 250 degrees. Referring to FIG. 4D, in one embodiment, it is desirable that the outer elongate instrument member (79) have a more flexible, steerable distal portion (109) approximately 2.5 centimeters in length (109), with a maximum bend angle (107) of approximately 120 degrees, defined as illustrated relative to the longitudinal axis of the unbent portion of the instrument member (79).

Referring to FIG. 4E, a side view of the inner elongate instrument housing assembly (77) is depicted, showing at least two control element interface assemblies (132) protruding downward for removable coupling with instrument driver sockets that are coupled with actuator motors move the control elements within the elongate instrument and cause controlled bending or steering, in response to control signals generated in response to operator actions at the operator workstation (for example, the master input device), as described in similar drive coupling configurations in the aforementioned incorporated by reference applications. In image capture device member (111) is removably coupled to the housing (77) to provide coupling of the light bundle and optics bundle to the housing, and ports are provided for integrating flush, laserfiber, working tools, and irrigation with the inner elongate instrument assembly. As shown in FIG. 4E, for example, a laser fiber interface, comprising an adjustable seal, is coupled to the housing (77), as is a tubular flush interface (115), and a working lumen interface comprising an adjustable seal arranged adjacent a purging port (172). Referring to FIG. 4F, a bottom portion of a inner elongate instrument base (132) partial assembly is depicted to show how the various components may be intercoupled, in a similar manner as described in the aforementioned incorporated by reference applications. For example, the inner instrument elongate member (81) is coupled to the inner elongate instrument base (132) assembly and has a proximally coupled working instrument port (170) with adjustable seal and purge port (172). Apertures (119) are defined within the wall of the inner elongate instrument member (81) to facilitate routing of the control elements (192) from their distal coupling locations within the elongate member (81), out the apertures, around pulleys comprising the control element interface assemblies (132) which also comprise drive axles configured to interface with instrument driver actuation sockets. Referring to FIGS. 4G and 4H, an embodiment of removable coupling between the image capture device/lighting member (111) is illustrated. Referring to FIG. 4G, the image capture device/lighting member (111) is inserted and twisted in a combined motion (183) into the interface (113) defined into the housing (77), whereby a BNC type mechanical coupling is formed utilizing fixtures and fittings on the outer surface of the image capture device/lighting member (111) and interface (113), as shown in FIG. 4H. FIG. 4I depicts a photograph of such a coupling being conducted with the help of an operator's hand. Referring to FIGS. 4J-4L, sterility issues associated with such removable coupling of the image capture device/lighting member (111) are illustrated. Generally the inner elongate instrument assembly, including the housing (77), will be sterile when unwrapped for a medical procedure. Referring to FIG. 4J, in one embodiment, sterility of the image capture device/lighting member (111) may be handled using a drape (123) having an optically translucent window (121) positioned to allow efficient optical signal transfer between the housing interface (113) and the image capture hardware (such as a camera chip) and lighting hardware (such as a fiber bundle termination) residing within the image capture device/lighting member (111); thus the image capture device/lighting member (111) may remain nonsterile. Referring to FIG. 4K, the drape (123) of FIG. 4J may be avoided with the depicted embodiment wherein the image capture device/lighting member (111) and associated elongate couplings (30, 32) are sterile (125). Referring to FIG. 4L, another draping (123) embodiment is depicted, but in this variation, rather than having a translucent window as in the embodiment of FIG. 4J, the nonsterile image capture device/lighting member (111) is simply kept out of contact with the housing (77) by virtue of a precision sterile fitting (127) configured to couple the drape, image capture device/lighting member (111), and housing interface (113) without allowing direct contact between the image capture device/lighting member (111) and the housing interface (113).

Referring to FIG. 5, in one embodiment it is desirable to have an inner elongate instrument assembly that is controllably lockable, such as those described in U.S. patent application Ser. No. 12/398,763, which is incorporated by reference in its entirety herein. The element labels of FIG. 5 correspond to those of this incorporated by reference application. Such a lockable embodiment may be useful and desirable, for example, after an instrument assembly has been advanced to a targeted tissue structure theater, such as the renal pelvis, to enable the operator to controllably lock one or more of the segments (90, 92, 94, 96, 98, for example) relative to each other, to provide a predictable conduit for the working tool or catheter assembly (88) which may be passed through the working lumen of such lockable catheter assembly and exposed distally (154), where the distal portion (154) may be utilized for diagnosis and/or intervention. Such a locked conduit may not only provide a predictable pathway for accessing the desired interventional theater, but also may decrease loads required to navigate working tools and/or catheters, and improve their steerability and maneuverability within such theather.

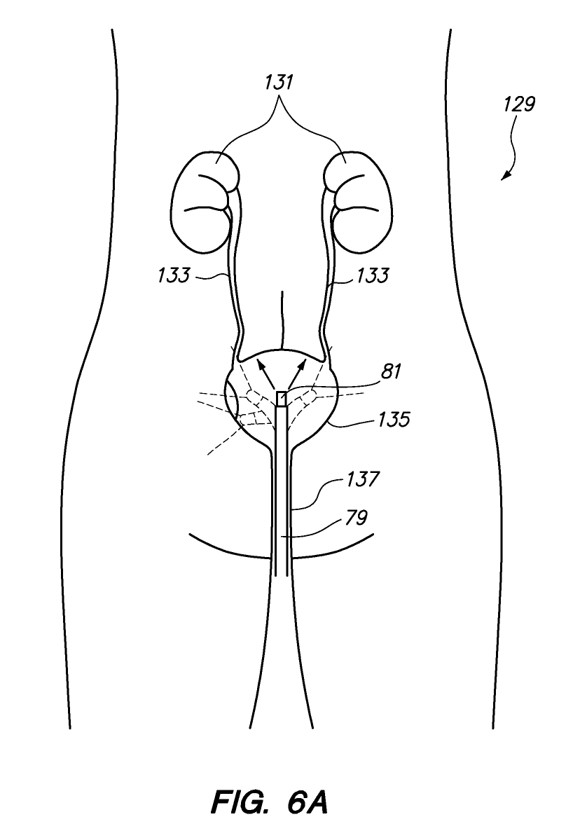

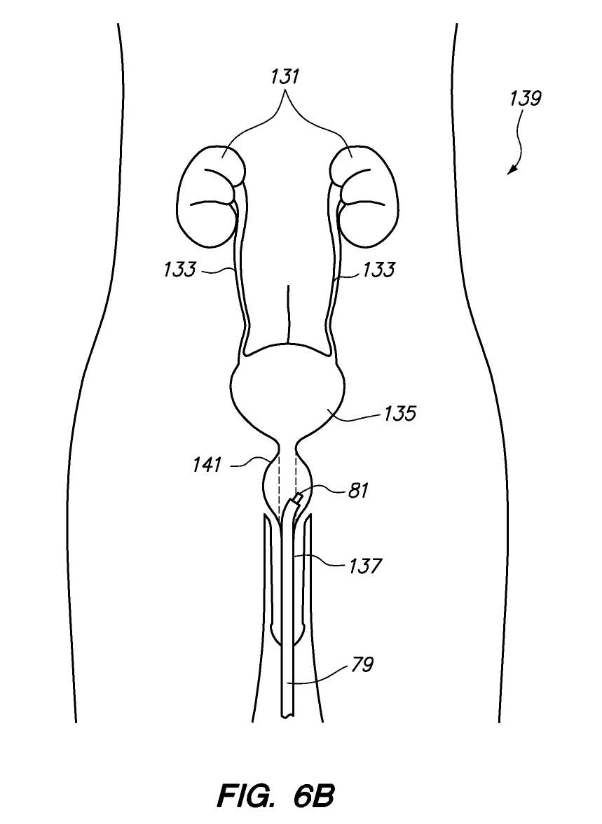

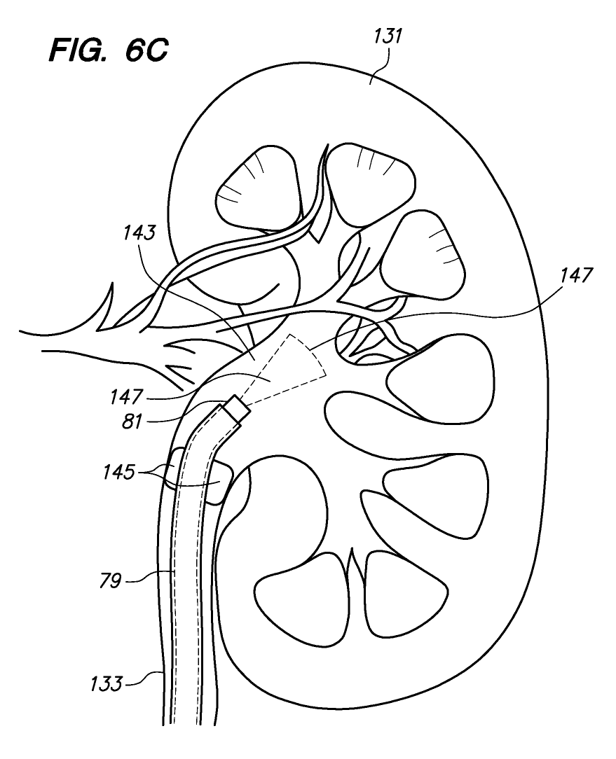

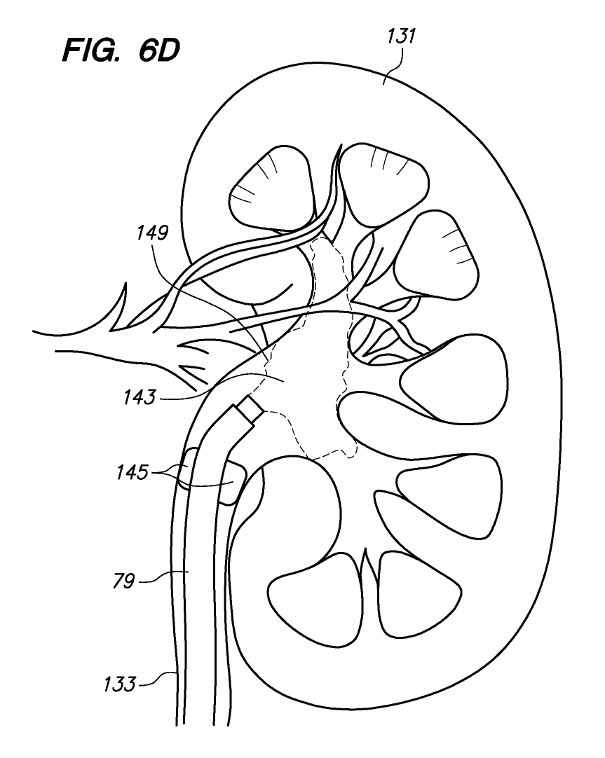

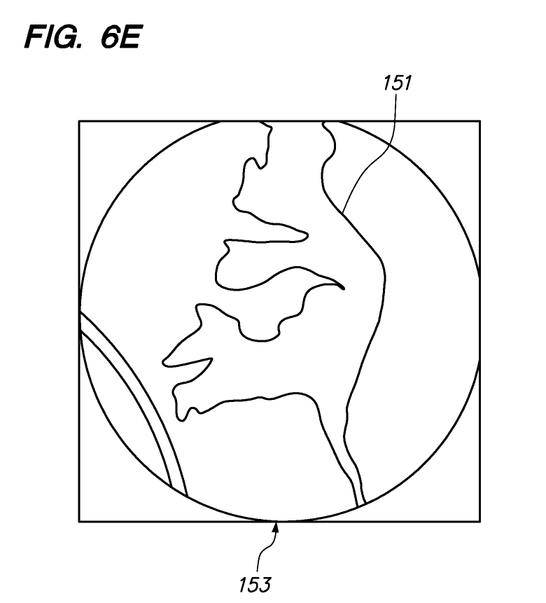

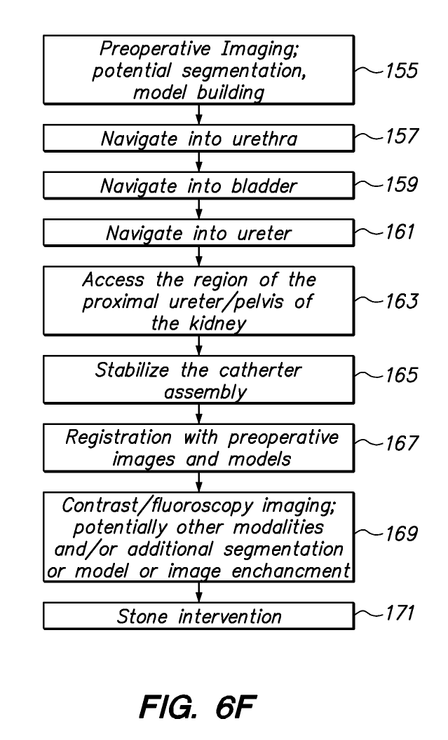

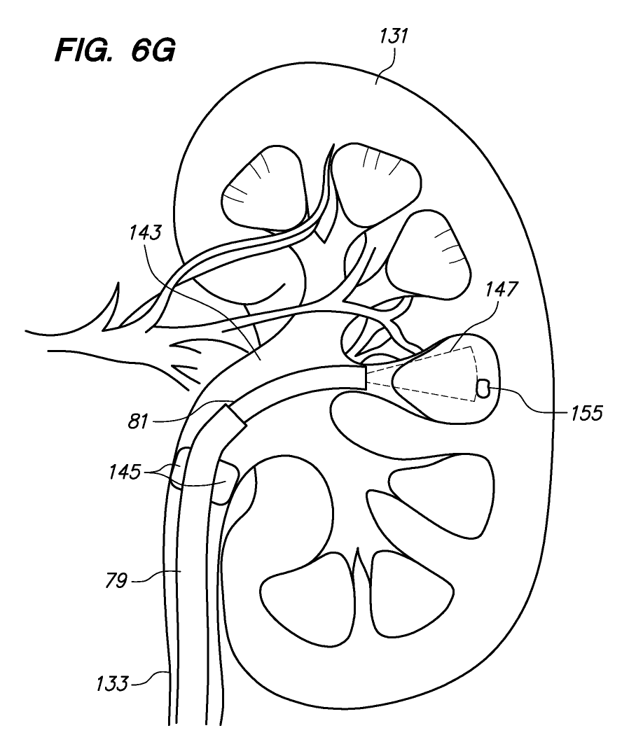

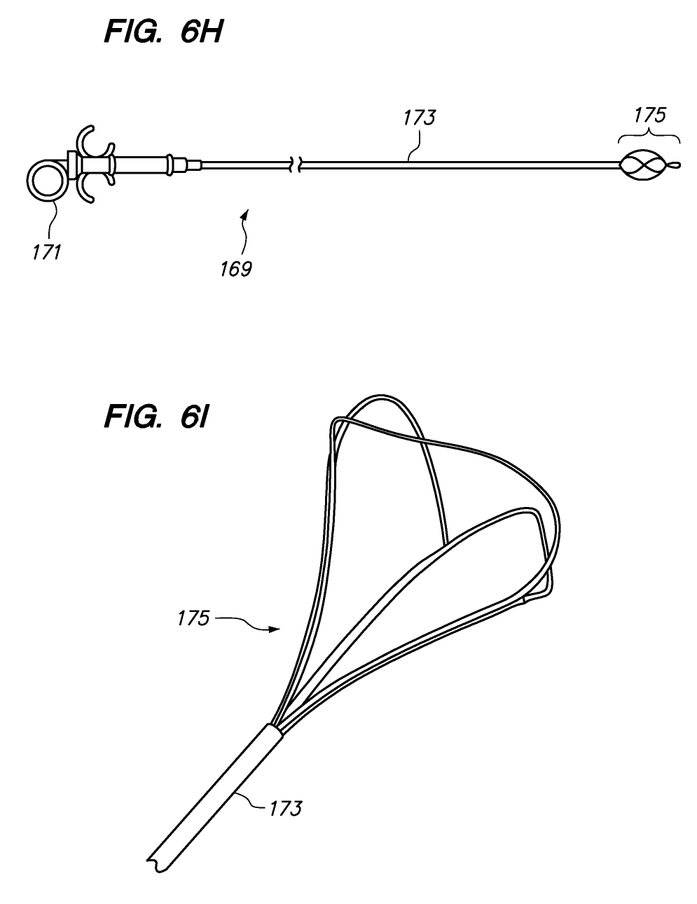

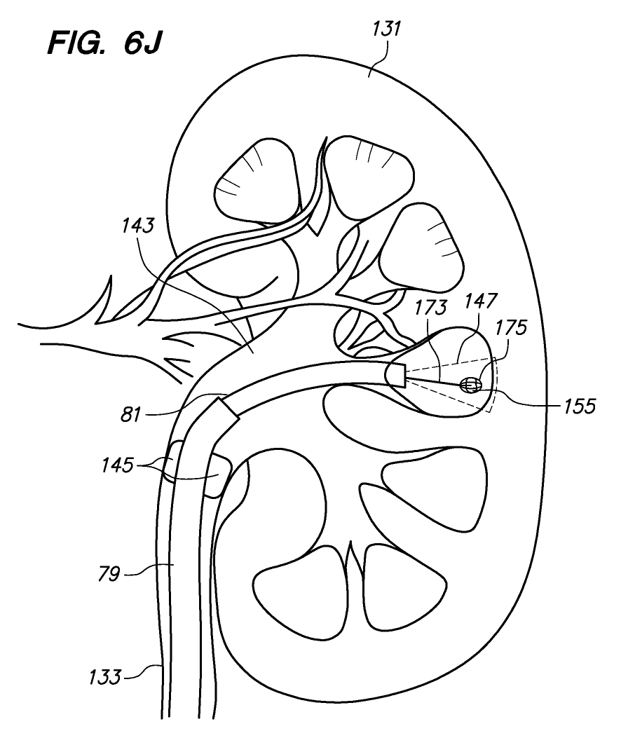

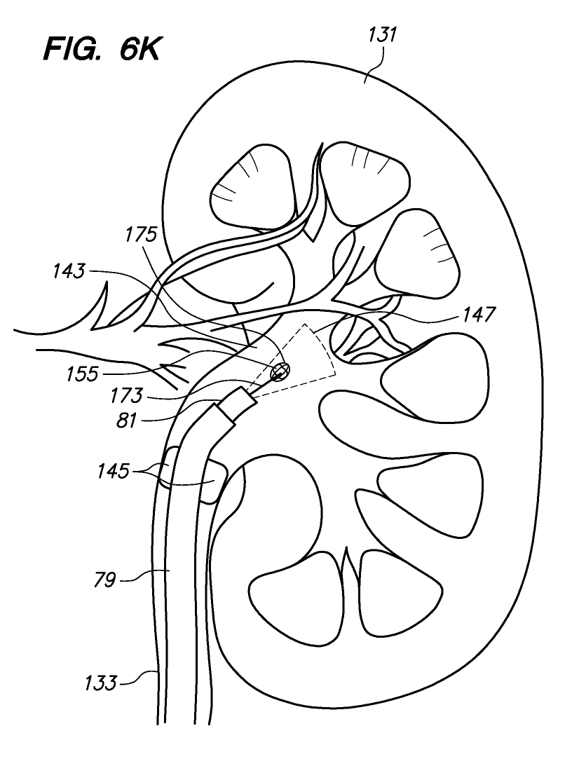

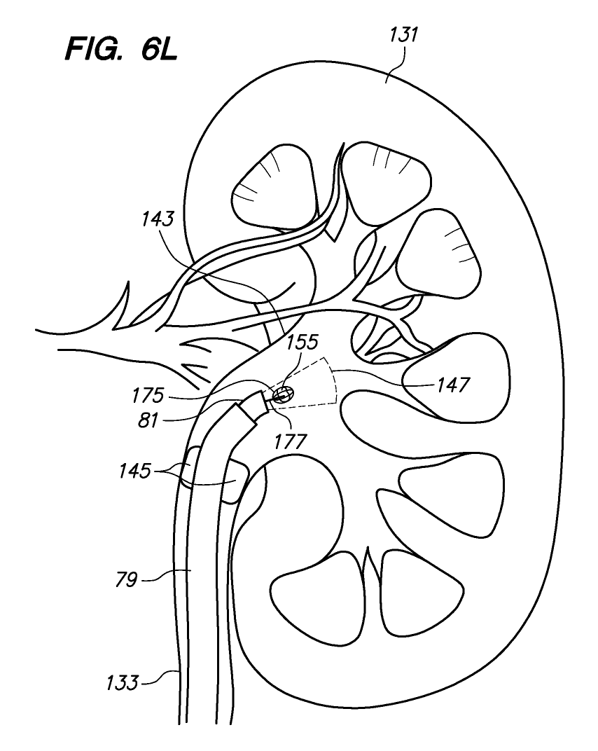

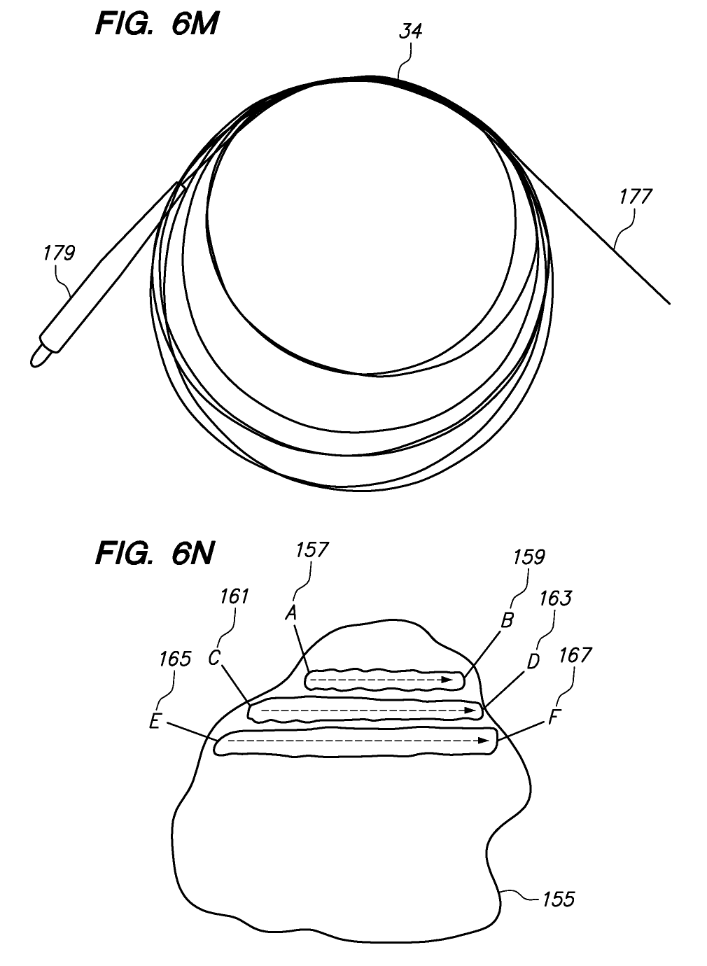

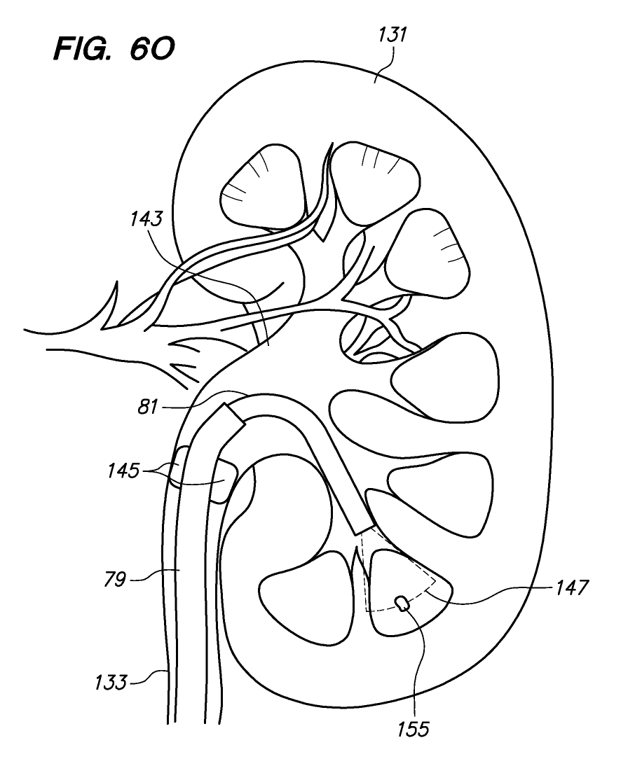

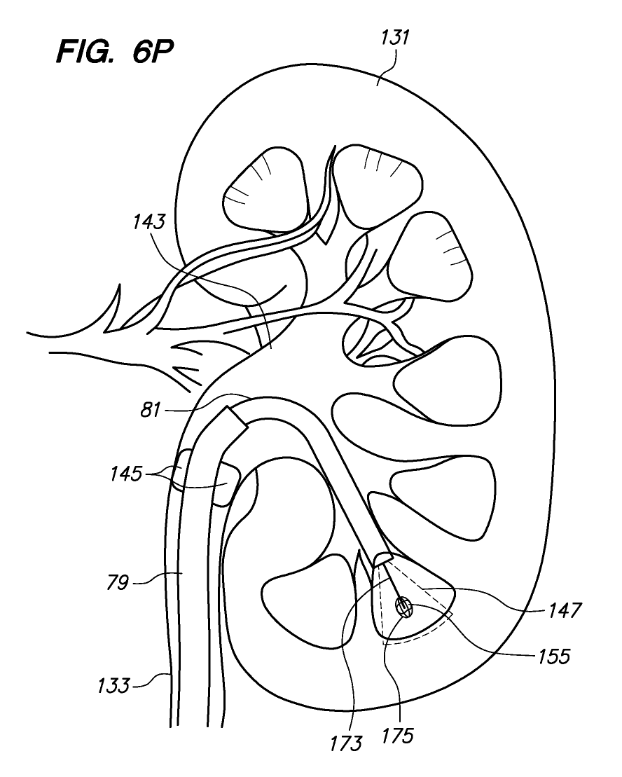

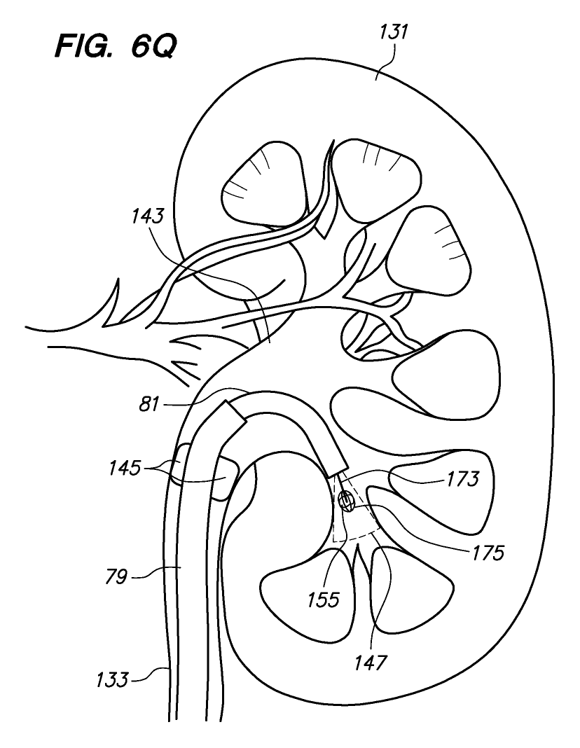

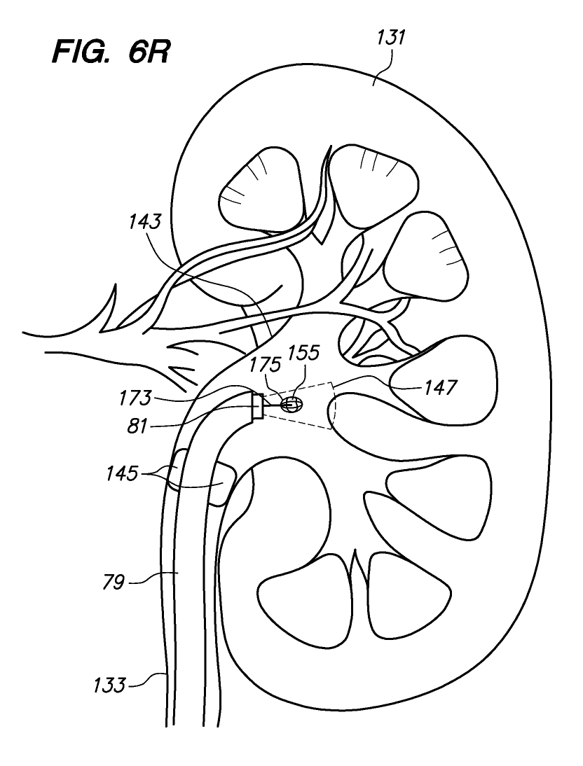

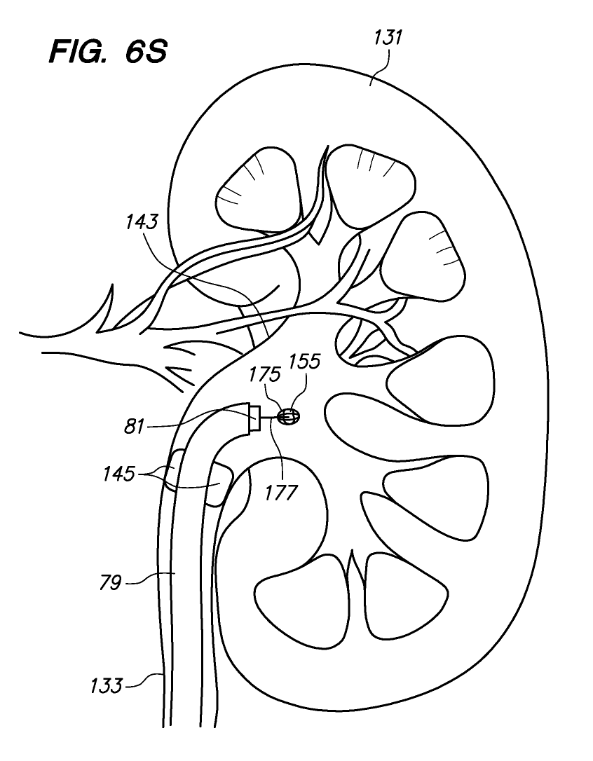

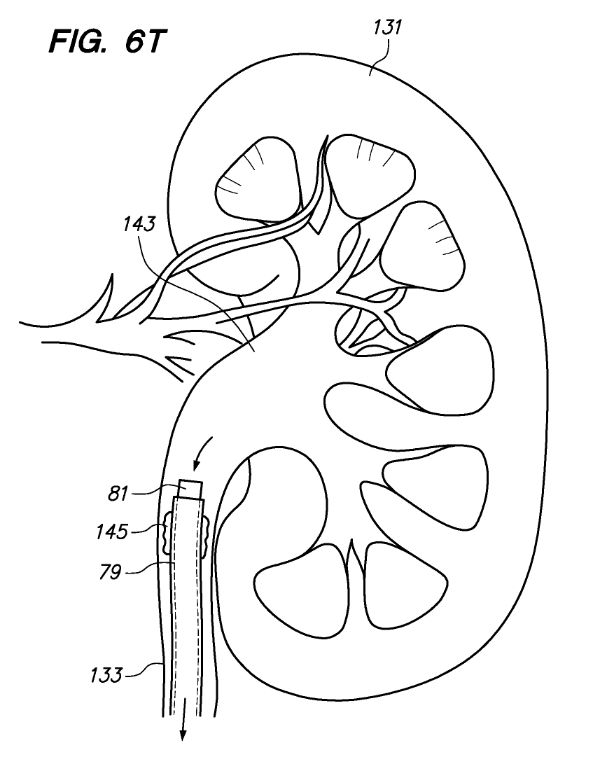

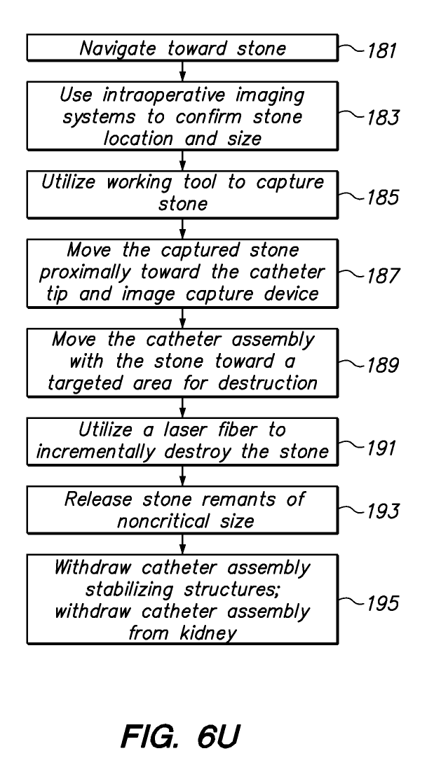

Referring to FIGS. 6A-6U, various aspects of diagnostic and/or interventional procedures and technologies are illustrated in reference to a urological clinical example. Referring to FIG. 6A, an inner instrument elongate body (81) and outer instrument elongate body (79), slidably assembled together, are inserted through the urethra of a female patient (129) to access the bladder. Referring to FIG. 6B, a similar instrument assembly is inserted through the urethra of a male patient, and through the prostate gland (141). Referring to FIG. 6C, the instrument assembly may be further advanced through the bladder, into one of the ureters (133) to access the pelvis (143) of the kidney (131). As shown in FIG. 6C, the optics bundle preferably has a forward-oriented field of view (147) with an included angle of about 80 degrees. Also shown in FIG. 6C is an expandable support member (145), such as an inflatable balloon, configured to controllably expand out against the walls of adjacent tissue structures and provide stability to the instrument assembly once the outer elongate instrument member (79) has been at least temporarily desirably placed (should movement be required, the operator may controllably retract or deflate such expandable support member 145). Referring to FIG. 6D, contrast agent (149) may be injected through the irrigation channel (95) and into the calices of the kidney (131) to facilitate fluoroscopic examination of the region. Referring to FIG. 6E, such a contrast agent utilization may reveal a geometric envelope (151) illustrating the volume of the inside of the kidney (131), as in the depicted fluoroscopy image (153). Utilizing bi-plane or multiplanar fluoroscopy images, a knowledge of the plane angles, and segmentation and registration techniques as described in the aforementioned incorporated by reference applications, the inside of the kidney may be turned into a mathematical model (for example, using a triangular mesh to model the surface), registered to the navigation environment and coordinate system of the robotic catheter assembly, and utilized for navigation of the instrumentation. Further imaging modalities may also be used preoperatively, intraoperatively, or postoperatively, as illustrated in the flow chart of FIG. 6F.

Referring to FIG. 6F, preoperative imaging, such as transcutaneous or endolumenal ultrasound, fluoroscopy, radiography, computed tomography, and/or magnetic resonance imaging may be utilized to generate images of pertinent anatomy, and these images maybe turned into models, voxel volumes, triangular mesh surfaces, and the like (155). The instrument system may be navigated into the urethra (157), then into the bladder (159), into the ureter (161), and to the kidney in the region of the pelvis of the kidney and/or proximal ureter (163). During all of this navigation, and during the entire procedure, for that matter, intraoperative imaging may be conducted to have confirmation regarding positioning of various instrumentation aspects, utilizing, for example, the light and imaging bundles on board the instrument assembly, fluoroscopy, ultrasound, etc. In the depicted embodiment, the catheter assembly, once in a desirable home position in the targeted kidney anatomic theater, may be stabilized (165), using, for example, a lockable spine mechanism or an expandable member such as a balloon. Preoperative and intraoperative images may be registered to the coordinate system of the robotic catheter assembly, so that the catheter may be navigated instinctively relative to such images and/or models (167). Newer intraoperative images may be utilized to improve upon or supplement the images, models, surfaces, etc derived from preoperative analysis, or earlier intraoperative activity (169). The system is then in a preferred configuration for a complex diagnostic or interventional process, such as a kidney stone intervention (171).

Referring to FIGS. 6G-6N, a kidney stone intervention in one of the middle calices of a kidney utilizing one embodiment of the subject invention is illustrated. Referring to FIG. 6G, the inner elongate instrument assembly (81) preferably is electromechanically advanced and navigated, subject to commands initiated by the operator at the operator workstation (for example, with a master input device or pendant) and executed by the computing system and instrument driver, toward the kidney stone (155) of interest. The forward-oriented field of view (147) of the optics bundle and lighting by the light bundle provide additional feedback to the operator through the displays on the operator workstation, which present images from the video system, as described in reference to FIG. 1. A working tool such as a collapsible basket tool may be utilized to capture stones. For example, referring to FIGS. 6H and 6I, a typical collapsible basket tool assembly (169) comprises a proximal actuation handle (171), a flexible elongate body (173), and a distally located, controllably collapsible, basket (175). Such tools are available from suppliers such as Boston Scientific Corporation, CR Bard Corporation, and Cook Medical, Inc. FIG. 6I depicts a close up view of the distally located basket (175). Referring to FIG. 6J, the basket (175) is advanced forward, preferably through the field of view (147), to capture the stone (155). Such advancing may be conducted by manual contact with the basket tool through the working lumen port of the inner elongate instrument assembly, or via operable coupling with an insertion/retraction and/or actuation means, such as a motor-based instrument actuator, as discussed in the above incorporated by reference applications and below as well. Referring to FIG. 6K, the basket (175) and inner elongate instrument assembly (81) are retracted proximally toward the outer elongate instrument assembly (79), and, as illustrated in FIG. 6L, the laser fiber tip (177) may be advanced forward (either manually or electromechanically, as described above in reference to the basket tool), preferably into the field of view (147) for optical confirmation of the intervention, and utilized to incrementally destroy the stone (155) via controlled emission of laser-based energy into the stone, preferably as controlled by the operator at the operator workstation using an input device such as a foot pedal, pendant button, or master input device command. FIG. 6M depicts a typical laser fiber (34) tool having a proximal coupling element (179) configured to interface with the laser system and a distal tip (177) configured to emit laser-based energy to destroy items such as kidney stones. Referring to FIG. 6N, preferably a stone is attacked with the laser tip in a pattern, to incrementally break the stone into smaller pieces of sub-critical geometry. For example, referring to FIG. 6N, a “painting” pattern may be utilized wherein the laser tip addresses the stone (155) from point A (157) to point B (159), then from point C (161) to point D (163), then from point E (165) to point F (167), and so on.

Referring to FIGS. 6O to 6T, another stone intervention embodiment is illustrated wherein the subject stone (155) is located in the lower pole of the kidney (131), as depicted in FIG. 6O. The inner elongate instrument (81) may be advanced toward the stone (155), captured with a working tool such as a grasper or basket tool, as shown in FIG. 6P, withdrawn toward a central working location, such as the pelvis of the kidney, as depicted in FIGS. 6Q and 6R, and incrementally destroyed with controlled advancement and actuation of the laser tip (177), as depicted in FIG. 6S. Subsequently, the entire tool assembly may be withdrawn, as depicted in FIG. 6T.

Referring to FIG. 6U, aspects of such intervention embodiments are illustrated in a flow chart. After an instrument is navigated toward a targeted stone (181), it may be captured with a working tool (185). Intraoperative imaging may be utilized throughout the procedure (183) for confirmation of relative locations of instruments, tissue structures, and the stone. The stone may be pulled proximally toward the inner elongate instrument tip (187), and the inner elongate instrument itself may be retracted toward the distal portion of the outer elongate instrument tip for an easier, and potentially safer, working environment (189) for destroying the stone incrementally (191). Stone remnants of noncritical size may be released or left in place (193), and the instrument assembly may be withdrawn from the kidney and ureters after collapsing, unlocking, and/or withdrawing any expanded or locked stabilizing structures.

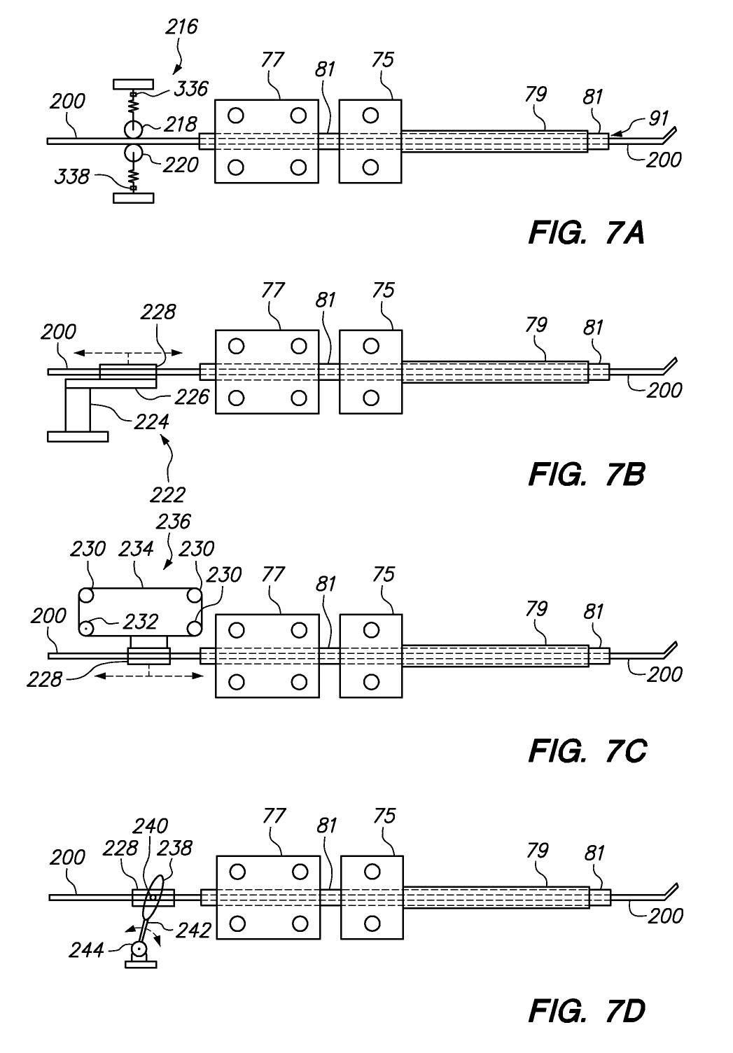

As described above, various medical instruments or tools, such as laser fibers, guidewires, collapsible basket tools, injection tools, and balloon inflation tools, may be electromechanically actuated to perform as part of the subject system. Such actuation may take the form of electromechanically assisted insertion and/or retraction, roll actuation, tool actuation, injectible actuation, and/or manual instrument interface actuation for one or more instruments, simultaneously. For illustrative purposes, embodiments of such actuation modes are illustrated serially in FIGS. 7A-12C. It is important to note that such modes may be combined to, for example, both controllably insert and roll a subject instrument.

Referring to FIGS. 7A-7D, four electromechanical insertion and/or retraction modalities are depicted for a tool, here a guidewire (200), in relation to a flexible catheter set (79, 81, 75, 77). Referring to FIG. 7A, a set of pinch roller wheels (218, 220) is interfaced with the proximally exposed aspect of the guidewire (200) and configured to insert or retract the guidewire (200) relative to the catheter set (79, 81, 75, 77) in accordance with rotational actuation imparted to one or both of the pinch roller wheels by one or two motors (not shown), each of which preferably is associated with an encoder to detect rotational motion with high precision. In an embodiment wherein one or both pinch roller wheels (218, 220) are rotationally actuated by motors, and rotation thereof is detected by two independent encoders, slippage may be detected as differences in the rotations, and insertion/retraction force imparted upon the guidewire (200) or other tool shaft may be backed out given a predetermined relationship between slippage and axial load. The pinch roller wheel assembly (216) preferably is mounted to an instrument driver or catheter instrument housing. In another embodiment wherein one pinch roller wheel is actively rotated and the other is passively rotated, encoders on both may similarly assist in the detection of slippage between the tool shaft and the pinch roller wheels to determine or estimate insertion/retraction load. In the depicted embodiment, load sensors (336, 338) assist in the sensing of forces applied normally to the surface of the guidewire, which also may be utilized in the aforementioned insertion/retraction force estimation technique, as friction forces are proportional to surface normal forces and friction coefficients. Insertion/retraction loads may also be estimated from predetermined relationships between applied motor current and insertion/retraction deflection. Such sensed information preferably is returned electronically to the controller, to enable load control loops, haptic feedback to the operator through a haptic master input device, safety monitoring, and other control and operational features, as described in the aforementioned incorporated by reference applications.

Referring to FIG. 7B, another embodiment is depicted featuring a lead screw assembly (222) to actuate insertion/retraction of a tool. As shown in FIG. 7B, a coupling sleeve (228) is configured to be removably coupled to a proximally exposed aspect of the tool, here a guidewire (200). Such removable coupling may be the result of changeable inner geometric features of the coupling sleeve (228), such as set screws, inflatable balloon lumens, electromagnetically movable locking features movable relative to the sleeve body, and the like. The sleeve (228) is coupled to a lead screw (226), which is coupled to a motor (224) and encoder (not shown) configured to monitor rotation of the motor and thereby translation of the lead screw, sleeve, and guidewire, in accordance with the pitch configuration of the lead screw (226). A load cell (not shown) may be utilized to sense insertion/retraction forces, or such loads may be backed out of the system utilizing relationships between motor current and insertion/retraction deflection.

Referring to FIG. 7C, a similar coupling sleeve (228) is depicted removably coupled to a proximally exposed aspect of a guidewire (220). Coupled to the sleeve to impart axial deflection is a recirculatory tension member (234), such as a belt or cable, which may be recirculated in one of two directions about a simple network of pulleys (230) and a motor capstan (232) directly coupled to a motor, and preferably an encoder to sense rotational positioning and thereby tension member (234), coupling sleeve (228), and tool shaft deflection. The depicted embodiment features an looped type recirculatory tension member (234) capable of being infinitely recirculated; in another embodiment, the recirculatory tension member (234) may comprise an incomplete loop, capable of a certain prescribed range of motion, but not infinite recirculation. A load cell (not shown) placed on a rotational idler pulley (230) or capstan (232) suspension may be used to sense tension in the tension member (234) and thereby insertion/retraction load. Loads may also be backed out of the system utilizing relationships between motor current and insertion/retraction deflection.

Referring to FIG. 7D, another coupling sleeve (228) is depicted removably coupled to a proximally exposed aspect of a guidewire (220). Coupled to the coupling sleeve is an exposed pin (240) configured to rotatably and slidably interface with a scotch yoke member (238) which is coupled to a rotating motor (244) by an arm member (242). As the motor is rotated clockwise or counterclockwise, insertion or retraction loads are imparted through the arm, yoke, pin, and sleeve to the guidewire (220). Load or torsion sensors, or motor current may be utilized to determine imparted insertion/retraction loads.

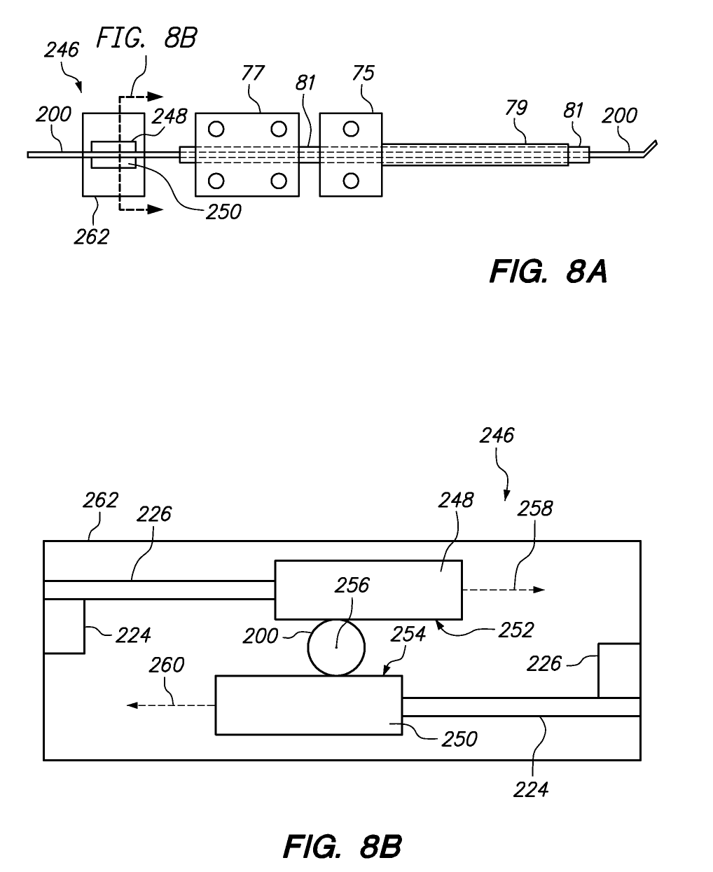

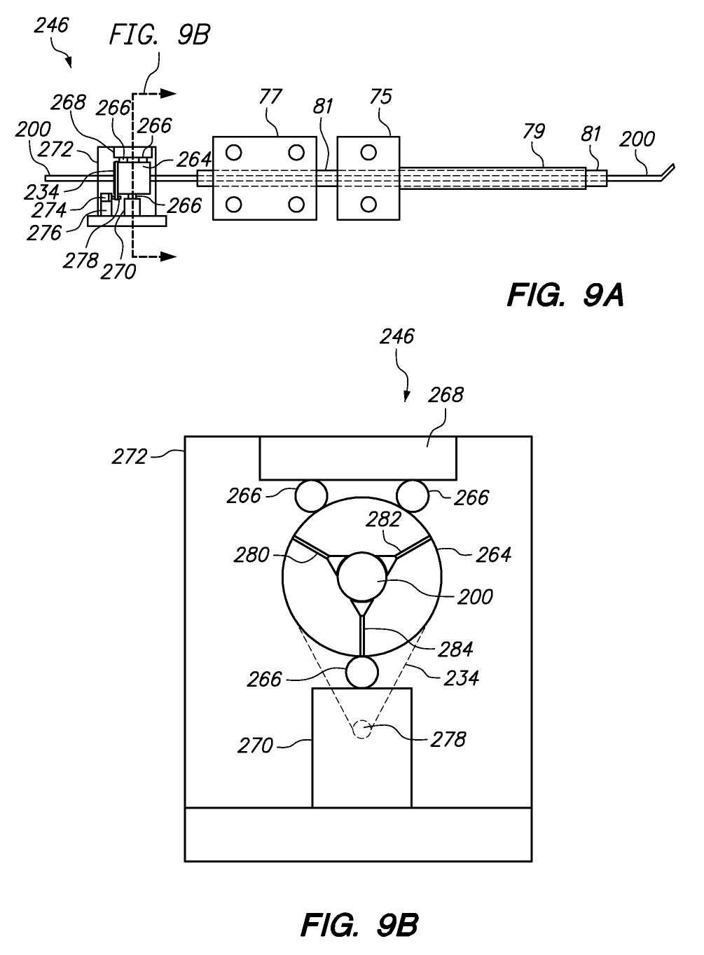

Referring to FIGS. 8A-9B, roll deflection, or rotational deflection, actuation configurations are depicted for elongate instruments placed through lumens of the above described steerable instrument assembly (79, 81, 75, 77). Referring to FIG. 8A, a configuration similar to that depicted in FIG. 7A is depicted, with the exception that a roll actuation assembly (246) is coupled to a proximally exposed aspect of the depicted guidewire (200). FIG. 8B illustrates a close up cross sectional view of the roll actuation assembly (246) coupled to the guidewire (200). Referring to FIG. 8B, two roll engagement members (248, 250) are moved (258, 260) in opposite directions in planes parallel to each other and parallel to the axis of roll rotation (256) of the guidewire (200) coupled between them. The deflections (258, 260) may both be actively actuated, as in the illustrated embodiment with two lead screw (224) and motor (226) assemblies coupled to the assembly (246) housing (262). Alternatively, one of these defections (258, 260) may be passive. The motors (226) preferably are associated with encoders to sense the deflections (258, 260). One or more load cells may be placed in line with one or both lead screws (224) to detect imparted loads, and motor currents also may be utilized in the determination of deflection load imparted. In the depicted embodiment, the roll engagement contract surfaces (252, 254) of the roll engagement members (248, 250) are planar. In another embodiment, they may be nonplanar surface portions of, for example, larger pulleys or capstans, such as those depicted in FIG. 7A.

Referring to FIG. 9A, another roll subassembly (246) embodiment is depicted coupled to the exposed proximal aspect of a guidewire (200). FIG. 9B illustrates a close up cross sectional view of the assembly (246) as interfaced with the tool shaft, here a guidewire (200) shaft. Referring to FIG. 9B, the guidewire shaft (200) preferably is removably coupled within a coupler (264) by a plurality of spaced apart coupling members (280, 282, 284) configured to be controllably engaged upon the guidewire shaft (200) using set screws, inflatable features, electromagnetically movable features, and the like. When engaged upon the guidewire shaft (200), rotation of the coupler (264) rotates the guidewire. The coupler (264) preferably is rotatably coupled to a housing (272) using a plurality of bearings (266) coupled in place relative to the housing by mounting members (268, 270), and may be rotationally actuated relative to the housing with a flexible tension member (234), such as a belt or cable, which preferably is routed around a capstan (278) and actuator motor/encoder assembly 274) also coupled (276) to the housing (272). Rotational loads or torques applied upon the shaft may be determined utilizing motor currents, encoder readings, and/or load cells associated with idler pulley (not shown) or capstan (278) suspension elements.

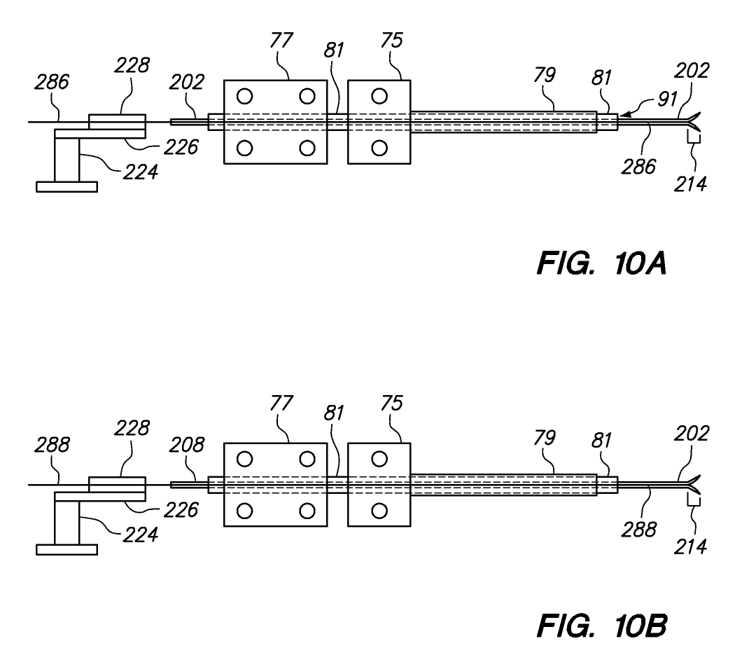

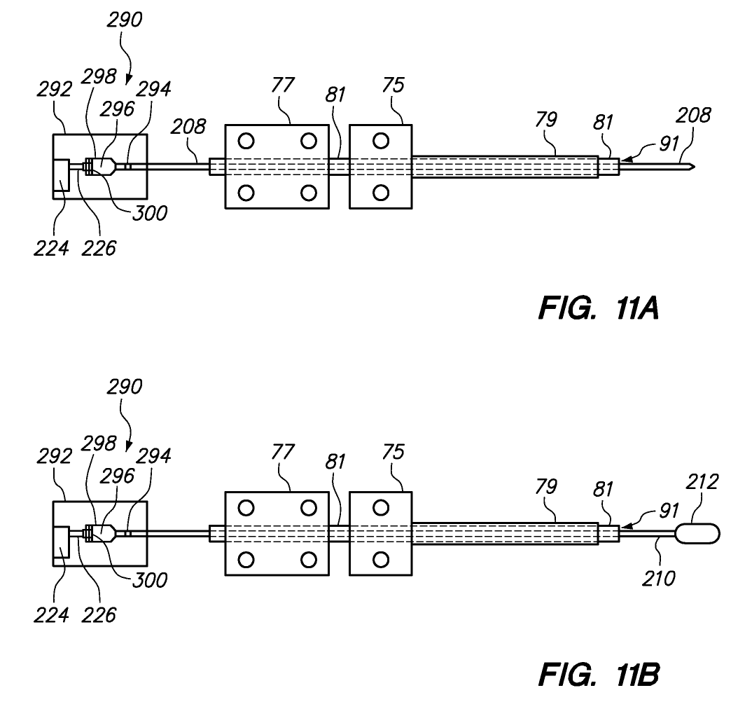

Referring to FIG. 10A, a grasper tool (202) is depicted positioned through a lumen of an instrument set (79, 81, 75, 77). The illustrated grasper tool (202) embodiment comprises a proximal shaft and a grasper end effector (214) that is spring-biased to stay in an open configuration unless closed by tension on a tension actuation member (288) that leads to a proximal exposure, as depicted in FIG. 10A. In another embodiment, a grasper tool spring biased to stay closed may be utilized, with a tension or compression actuation member configured to open or close the grasper end effector; similarly, in other embodiments, scissors and other tools may be actuated. Referring to FIG. 10A, the tension actuation member (286) may be inserted or retracted, for example, in accordance with each of the insertion/retraction embodiments described in reference to FIGS. 7A-7D. A motorized (224) lead screw (226) variation is depicted in FIG. 10A. FIG. 10B depicts a similar embodiment, wherein the grasper tool (202) end effector (214) is spring biased to stay closed unless pushed open with a compression actuation member (288), such as an elongate push rod. As shown in FIG. 10B, the compression actuation member may be electromechanically inserted or retracted, for example, in accordance with the insertion/retraction embodiments described in reference to FIGS. 7A-7D. A motorized (224) lead screw (226) variation is depicted in FIG. 10B.

Referring to FIGS. 11A and 11B, injection actuation embodiments are depicted, wherein an electromechanical subassembly is utilized to controllably inject fluid from a reservoir (296) to the distal end of an instrument, which in FIG. 11A comprises an injection needle (208), and in FIG. 11B comprises an inflatable balloon (212) similar to the expandable stabilizer (145) described above, but located at the distal tip of the illustrated elongate instrument. Injectable fluids may comprise contrast agents, saline, medicines or treatment solutions, inert gases, carbon dioxide, and the like. Preferably the injection actuation assembly (290) comprises a fluid reservoir (296) encapsulated by a reservoir structure (298) and a piston (300), the piston being electromechanically movable relative to the reservoir structure (298) by a translation actuation means, such as those described in reference to FIGS. 7A-7D, to produce increased or decreased pressure in the reservoir, and thereby variations of injection or suction at the distal end of the instrument through a fluid coupling (via lumens defined in tubular couplings and the elongate instrument body) between the reservoir and the distal aspect of the instrument. A motorized (224) lead screw (226) assembly is shown in each of the assemblies in FIGS. 11A and 11B. Preferably a pressure sensor (294) is located in line with the fluid coupling between the reservoir (296) and distal portion to detect output pressures from the piston (300) and reservoir structure (298) configuration, to allow for closed loop control of applied injection or suction profiles versus time, utilizing the computerized system controller. Precise volumes and pressure versus time profiles may be produced with such system, to allow for precision injection of contrast agent, medicines, saline, gases, and the like, as described above.

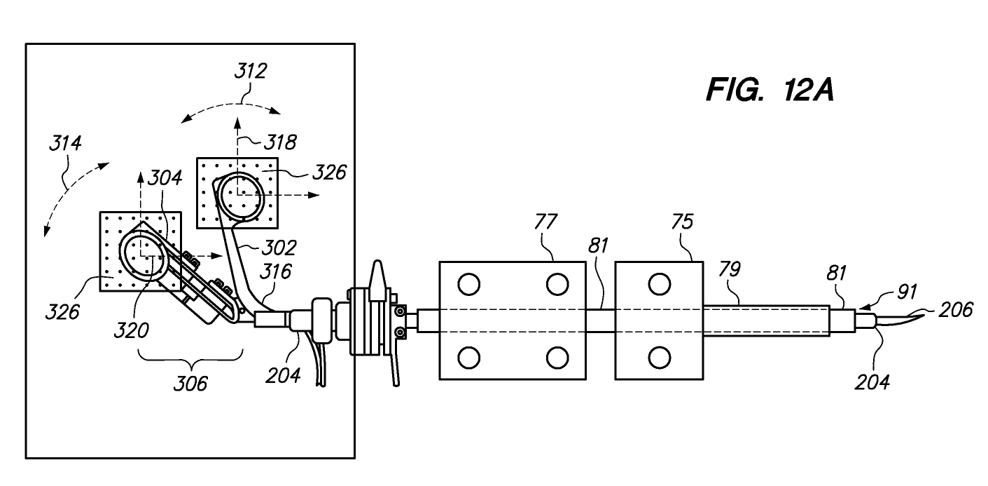

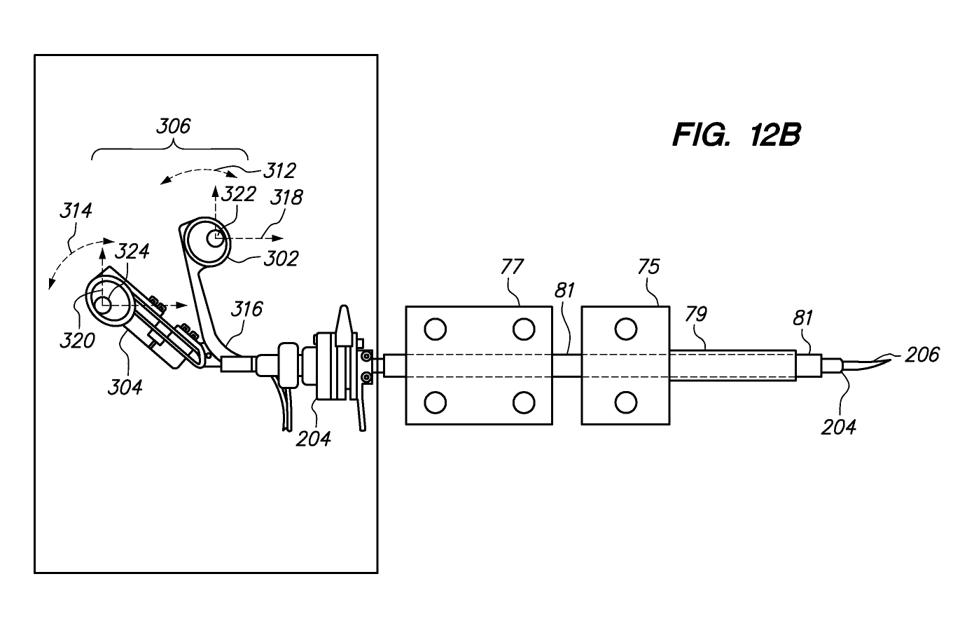

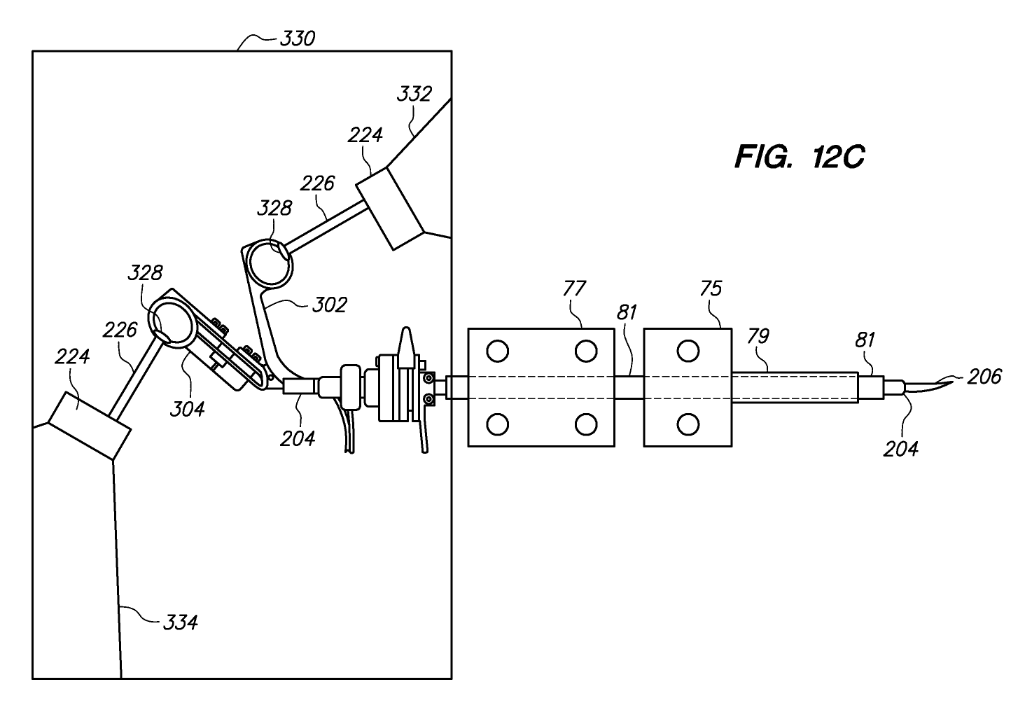

Referring to FIGS. 12A-12C, embodiments for electromechanically actuating proximal interfaces configured for manual (i.e., by human hand) actuation. Referring to FIGS. 12A-12C, a conventional endoscopic tool comprising a dissection scissor tip (206) and proximal manual actuation interface (306) is depicted threaded through a steerable instrument assembly (79, 81, 75, 77). Other manual actuation interfaces (i.e., other than the scissor type finger eyelets depicted in FIGS. 12A-12C), such as knobs, sliding interfaces, and the like, may also be electromechanically actuated utilizing these techniques. Referring to FIG. 12A, two substantially square pin matrix platforms (326), similar to those available from manufacturers such as Endeavor Tool Company of Boylston, Mass., under the tradename, “Gator Grip”, may be pressed against and conformed around the two handle portions (302, 304) in a direction substantially orthogonal to the desired plane of rotation (312, 314) relative to an axis of rotation (316) of the handle portions (302, 304), or Cartesian translation (318, 320) of the handle portions (302, 304). Preferably the Cartesian (318, 320) and/or rotational (312, 314) motion imparted to the handle portions (302, 304) is conducted with one of the aforementioned electromechanical displacement configurations. For example, in one embodiment, independent Cartesian translation (318, 320) of each handle portion (302, 304) is produced approximately in the plane of the page of FIGS. 12A and 12B utilizing a stack of two motorized lead screw assemblies to produce X-Y deflection. Rotational motion (312, 314) may be actuated with two independent motors having rotational axes approximately coincident with the axis of rotation of the handle portions (302, 304), and arm members (not shown) coupling the motors to the pin platforms (326). In some embodiments, it is desirable to provide such rotational motion using a torquable coupling interposed between a motor interface and the instrument interface that is specifically configured to allow for some misalignment between the axis of rotation of the actuator motor and the axis of rotation of the fitting associated with the instrument interface, such as a Schmidt type mechanical movement coupling. FIG. 12B depicts an embodiment wherein simple post elements (322, 324) are utilized rather than pin arrays to engage the handle portions (302, 304). FIG. 12C depicts an embodiment wherein a separate motorized (224) lead screw (226) assembly is coupled between a housing (330) and each of the handle portions (302, 304) for direct engagement, via a pair of coupling FIGS. 328) and electromechanical actuation of the tool.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A robotic catheter system, comprising:
a. a robotically steerable catheter instrument comprising a housing and an elongate flexible body, the elongate flexible body defining one or more instrument lumens longitudinally therethrough;
b. an electromechanical instrument driver comprising a housing, a plurality of motors, and an instrument interface configured to removably engage the catheter instrument housing and transfer a plurality of steering actuations to the catheter instrument when engaged thereto;
c. an elongate medical instrument slidably positioned through one of the one or more instrument lumens;
d. an electromechanical device actuation assembly coupled to the proximal end of the elongate medical instrument and configured to actuate the medical instrument independently of steering actuations transferred to the robotically steerable catheter instrument; and
e. a controller configured to operate the instrument driver and device actuation assembly to respectively steer the catheter instrument and actuate the elongate medical instrument subject to commands imparted by an operator to a master input device operably coupled to the controller.

2. The catheter system of claim 1, wherein the one or more instrument lumens are configured to separately and simultaneously accommodate an optics bundle, a light bundle, and a laser fiber, and to define a flush channel.

3. The catheter system of claim 1, wherein the instrument housing comprises an optics bundle connection interface.

4. The catheter system of claim 1, wherein the instrument housing comprises a light bundle connection interface.

5. The catheter system of claim 1, wherein the instrument housing comprises a laser fiber coupling interface.

6. The catheter system of claim 1, wherein the instrument housing comprises a flush channel supply interface.

7. The catheter system of claim 1, wherein one of the one or more instrument lumens is sized to accommodate slidable coupling of a collapsible basket tool.

8. The catheter system of claim 4, wherein the optics bundle connection interface comprises a translucent light transmission interface between fibers comprising the optics bundle in the housing and fibers comprising an external optics bundle coupled to a video processing device.

9. The catheter system of claim 4, wherein the optics bundle connection interface comprises a translucent light transmission interface between fibers comprising the optics bundle in the housing and a digital image capture chip.

10. The catheter system of claim 1, wherein the elongate flexible body comprises a substantially square cross sectional shape having corners.

11. The catheter system of claim 10, wherein the corners are rounded in shape.

12. The catheter system of claim 10, wherein the elongate flexible body comprises a polymeric coextrusion.

13. The catheter system of claim 10, wherein the elongate flexible body defines four integrated instrument lumens arranged immediately adjacent the corners.

14. The catheter system of claim 13, wherein the elongate flexible body defines four control wire lumens arranged between the four integrated instrument lumens.

15. The catheter system of claim 14, wherein the working instrument lumen has a diameter which is greater than that of the integrated instrument lumens, and wherein the control wire lumens have a diameter which is smaller than that of the integrated instrument lumens.

16. The catheter system of claim 2, wherein the elongate body has a first portion with a first flexibility and a second portion extending distally from the first portion, the second portion having a second flexibility substantially greater than the first flexibility.

17. The catheter system of claim 2, further comprising a flexible sheath body defining a working lumen sized to accommodate slidable engagement of the elongate flexible body, and an outer cross sectional shape that is substantially circular.

18. The catheter system of claim 17, wherein the shape of the working lumen of the sheath body and outer shape of the elongate flexible body are jointly configured to limit rotational movement of the elongate flexible body relative to the sheath body.

19. The catheter system of claim 1, wherein the device actuation assembly is configured to controllably insert and retract the medical instrument relative to the catheter instrument.

20. The catheter system of claim 19, wherein the device actuation assembly comprises two or more pinch roller wheels movably coupled to the medical instrument.

21. The catheter system of claim 19, wherein the device actuation assembly comprises a lead screw assembly coupled to the medical instrument.

22. The catheter assembly of claim 19, wherein the device actuation assembly comprises a recirculatory tension member assembly movably coupled to the medical instrument.

23. The catheter assembly of claim 19, wherein the medical instrument is selected from the group consisting of a laser fiber, a guidewire, a collapsible basket tool, a flexible needle, a grasper, and a balloon tool.

24. The catheter system of claim 1, wherein the device actuation assembly is configured to controllably roll the medical instrument relative to the catheter instrument.

25. The catheter system of claim 24, wherein the device actuation assembly comprises two surfaces configured to engage opposite sides of the medical instrument, and to roll the medical instrument as at least one of such surfaces is moved relative to each other through a motion plane substantially parallel to an axis of roll motion defined as the medical instrument is rolled.

26. The catheter system of claim 25, wherein one of the two surfaces is substantially planar.

27. The catheter system of claim 25, wherein one of the two surfaces comprises a pinch roller wheel surface.

28. The catheter system of claim 24, wherein the device actuation assembly comprises a rolling subassembly that is coupled to a coupling element configured to removably and fixedly couple to the medical instrument, and wherein the rolling subassembly is rotatably coupled to a base element configured to not rotate relative to the instrument driver.

29. The catheter system of claim 24, wherein the medical instrument is selected from the group consisting of a laser fiber, a guidewire, a collapsible basket tool, a flexible needle, a grasper, and a balloon tool.

30. The catheter system of claim 1, wherein the device actuation assembly comprises an elongate tension element operably coupled between an actuator coupled to a housing of the device actuation assembly and a distally operable element of the medical device such that activation of the actuator creates tension in the tension element and a pulling actuation of the distally operable element.

31. The catheter system of claim 30, wherein the distally operable element is selected from the group consisting of a grasper closure element, a grasper opener element, a collapsible basket collapse element, and a collapsible basket expand element.

32. The catheter system of claim 1, wherein the device actuation assembly comprises an elongate compression element operably coupled between an actuator coupled to a housing of the device actuation assembly and a distally operable element of the medical device such that activation of the actuator creates compression in the tension element and a pushing actuation of the distally operable element.

33. The catheter system of claim 32, wherein the distally operable element is selected from the group consisting of a grasper closure element, a grasper opener element, a collapsible basket collapse element, and a collapsible basket expand element.

34. The catheter system of claim 1, wherein the device actuation assembly comprises an injection pump fluidly coupled with one of the lumens defined through the elongate flexible body and configured to inject or suction out fluid through said lumen.

35. The catheter system of claim 34, wherein the injection pump comprises a piston operably coupled to an actuator and movably coupled to a fluid reservoir housing.

36. The catheter system of claim 34, wherein the injection pump comprises a reservoir configured to contain an injectible fluid selected from the group consisting of liquid contrast agent, saline, and liquid therapeutics.

37. The catheter system of claim 34, wherein the medical instrument comprises a balloon tool or a flexible needle.

38. The catheter system of claim 1, wherein the medical instrument has a proximal end comprising a manual control interface, and wherein the device actuation assembly comprises one or more fittings configured to removably couple to one or more portions of the manual control interface to substantially reproduce relative motions of such portions when operated manually.

39. The catheter system of claim 38, wherein at least one of said one or more fittings comprises a pin array.

40. The catheter system of claim 38, wherein at least one of said one or more fittings comprises a post array.

41. The catheter system of claim 38, wherein at least one of said one or more fittings is coupled to a motion subassembly selected from the group consisting of an assembly of one or more leadscrews and a recirculatory tension member assembly.

42. The catheter system of claim 38, wherein at least one of said one or more fittings is coupled to an assembly of two lead screws configured to move orthogonally relative to each other.

43. The catheter system of claim 38, wherein at least one of the one or more fittings is configured to be electromechanically rotated relative to other portions of the manual control interface.

44. The catheter system of claim 43, wherein the device actuation assembly comprises a torquable coupling configured to allow for misalignment between an axis of rotation of a rotation actuator providing electromechanical rotation and an axis of rotation of the fitting configured to be electromechanically rotated.

45. The catheter system of claim 44, wherein the torquable coupling comprises a Schmidt type coupling.
